(19) United States
(12) Patent Application Publication
(10) Pub. No.: US 2008/0311076 A1
(43) Pub. Date: Dec. 18, 2008

(54) Morpholinylanilinoquinazoline Derivatives For Use As Antiviral Agents

(75) Inventors: Keith Charles Spencer, London (GB); Helena Dennison, London (GB); Neil Mathews, London (GB); Michael Christopher Barnes, London (GB); Surinder Singh Chana, London (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: ARROW THERAPEUTICS LIMITED, London (GB)

(21) Appl. No.: 11/587,687

(22) PCT Filed: Apr. 28, 2005
(86) PCT No.: PCT/GB2005/001598

§ 371 Date: Oct. 26, 2006

(30) Foreign Application Priority Data
Apr. 28, 2004 (GB) 0409494.2
Nov. 16, 2004 (GB) 0425268.0

(51) Int. Cl. A61K 38/21; C07D 413/02; A61K 31/5377; C07D 417/14; A61P 31/12; A61K 31/7056; A61K 31/541; C07D 413/14

(52) U.S. Cl. 424/85.4; 544/119; 514/234.5; 544/62; 514/228.2; 544/80; 514/232.5; 514/43

(57) ABSTRACT

Compounds of formula (Ia) are found to be active in inhibiting replication of flaviviridae viruses, wherein R₁, R₂, R₃ and R⁴ are as defined in the claims.

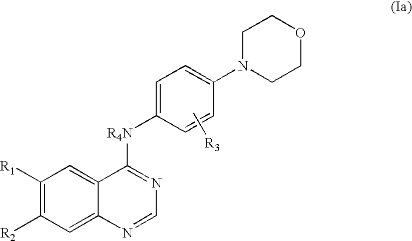

The present invention relates to a series of quinazoline derivatives which are useful in treating or preventing a flaviviridae infection.

Viruses of the family flaviviridae are small, icosahedral, enveloped viruses that contain a positive-sense RNA genome. The family consists of three genera, flavivirus, pestivirus and hepacivirus.

Many of the flaviviridae viruses are important human pathogens. Indeed, the hepacivirus genus includes the hepatitis C virus. However, there exists, as yet, no effective and safe treatment for flaviviridae infections.

WO 98/02434 discloses quinazolines as protein tyrosine kinase inhibitors. None of the compounds specifically disclosed in that document carry a morpholino-aniline-group at the 6-position.

It has now surprisingly been found that the quinazoline derivatives of the formula (Ia) are active in inhibiting replication of flaviviridae viruses and are therefore effective in treating or preventing a flaviviridae infection. The present invention therefore provides a quinazoline derivative of formula (Ia), or a pharmaceutically acceptable salt thereof.

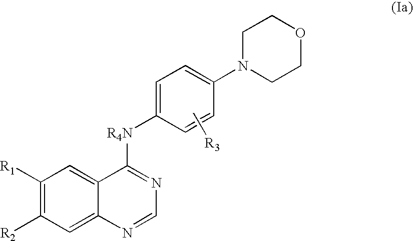

wherein R₁ represents hydrogen, halogen, C₁-C₄ alkyl, C₁-C₄ haloalkyl, C₁-C₄ alkoxy, C₁-C₄ haloalkoxy, —CO₂R′, —CONR′R″, -A, -A-L-A′, -Z-L-A or -A-L-Z-L-A, wherein R′ and R″ are the same or different and each represent hydrogen or C₁-C₄ alkyl;

R₂ represents hydrogen, halogen, C₁-C₄ alkyl, C₁-C₄ haloalkyl, C₁-C₄ alkoxy or C₁-C₄ haloalkoxy;

R₃ represents hydrogen, C₁-C₄ alkyl, C₁-C₄ haloalkyl, C₁-C₄ alkoxy or C₁-C₄ haloalkoxy; and

R₄ represents hydrogen, C₁-C₆ alkyl or C₁-C₆ haloalkyl, wherein:
A represents a C₆ to C₁₀ aryl, 5- to 10-membered heteroaryl or 5- to 10 membered heterocyclyl group;
each L is the same or different and is a direct bond or a C₁-C₄ alkylene group;
A′ is a 5- to 10-membered heteroaryl or 5- to 10-membered heterocyclyl group; and
Z is —S—, —O—, —NR′—, —CO₂—, —C(O)NR′—, —OC(O)—, —NR′C(O)—, —OCO₂—, —NR′CO₂—. —OC(O)NR′—, or —NR′C(O)NR″—, wherein R′ and R″ are the same or different and represent hydrogen or C₁-C₄ alkyl,

the aryl, heteroaryl and heterocyclyl moieties in R₁ being unsubstituted or substituted by 1, 2 or 3 substituents selected from halogen, C₁-C₄ alkyl, C₁-C₄ haloalkyl, C₁-C₄ haloalkoxy, hydroxy, thiol, —NH₂, C₁-C₄ hydroxyalkyl, C₁-C₄ thioalkyl, C₁-C₄ aminoalkyl, cyano, nitro, —COR′, —CO₂R′, —S(O)R′, —S(O)₂R′, —CONR′R″ and -L′-X-L″-Y substituents, wherein each R′ and R″ is the same or different and is selected from hydrogen and C₁-C₄ alkyl, L′ is a direct bond or a C₁-C₄ alkylene group, X is —S—, —O— or —NR′— wherein R′ is as defined above, L″ is a direct bond or a C₁-C₄ alkylene group and Y is hydrogen, —COR′, —CO₂R′, —S(O)₂R′, or —S(O)R′, wherein R′ is hydrogen or C₁-C₄ alkyl.

For the avoidance of doubt, the orientation of the Z moiety is such that the left hand side of the depicted groups is attached to the quinazoline group or to the -A-L-moiety. Thus, for example, when Z is —C(O)NR′— and R¹ is -Z-L-A, R¹ is —C(O)NR′-L-A.

In one embodiment, the quinazoline derivative of formula (Ia) is a quinazoline derivative of formula (I),

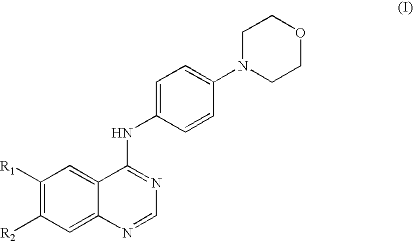

wherein R₁ represents hydrogen, halogen, C₁-C₄ alkyl, C₁-C₄ haloalkyl, C₁-C₄ alkoxy, C₁-C₄ haloalkoxy, -A or -A-L-A′ and R₂ represents hydrogen, halogen, C₁-C₄ alkyl, C₁-C₄ haloalkyl, C₁-C₄ alkoxy or C₁-C₄ haloalkoxy, wherein:
A represents a C₆ to C₁₀ aryl, 5- to 10-membered heteroaryl or 5- to 10-membered heterocyclyl group;
L is a direct bond or a C₁-C₄ alkylene group; and
A′ is a 5- to 10-membered heteroaryl or heterocyclyl group,

the aryl, heteroaryl and heterocyclyl moieties in R₁ being unsubstituted or substituted by 1, 2 or 3 substituents selected from halogen, C₁-C₄ alkyl, C₁-C₄ haloalkyl, C₁-C₄ haloalkoxy, hydroxy, thiol, —NH₂, C₁-C₄ hydroxyalkyl, C₁-C₄ thioalkyl, C₁-C₄ aminoalkyl, cyano, nitro, —COR′, —CO₂R′, —CONR′R″, —SOR′, —S(O)₂R′ and -L′-X-L″-Y substituents, wherein each R′ and R″ is the same or different and is selected from hydrogen and C₁-C₄ alkyl, L′ is a direct bond or a C₁-C₄ alkylene group, X is —S—, —O— or —NR′— wherein R′ is as defined above, L″ is a C₁-C₄ alkylene group and Y is hydrogen, —COR′, —CO₂R′, —S(O)₂R′, or —S(O)R′, wherein R′ is hydrogen or C₁-C₄ alkyl.

Typically, the aryl, heteroaryl and heterocyclyl moieties in R₁ in the formula (I) are unsubstituted or substituted by 1, 2 or 3 substituents selected from halogen, C₁-C₄ alkyl, C₁-C₄ haloalkyl, C₁-C₄ haloalkoxy, hydroxy, thiol, —NH₂, C₁-C₄ hydroxyalkyl, C₁-C₄ thioalkyl, C₁-C₄ aminoalkyl, cyano, nitro, —COR′, —CO₂R′, —CONR′R″ and -L′-X-L″-Y substituents, wherein each R′ and R″ is the same or different and is selected from hydrogen and C₁-C₄ alkyl, L′ is a direct bond or a C₁-C₄ alkylene group, X is —S—, —O— or —NR′— wherein R′ is as defined above, L″ is a C₁-C₄ alkylene group and Y is hydrogen, —COR′, —CO₂R′, —S(O)₂R′, or —S(O)R′, wherein R′ is hydrogen or C₁-C₄ alkyl.

As used herein, a C₁-C₆ alkyl group or moiety is a linear or branched alkyl group or moiety containing from 1 to 6 carbon atoms. Typically a C₁-C₆ alkyl group or moiety is a C₁-C₄ alkyl group or moiety. A C₁-C₄ alkyl group or moiety is a linear or branched alkyl group or moiety containing from 1 to 4 carbon atoms. Examples of C₁-C₆ alkyl groups and moieties include methyl, ethyl, n-propyl, i-propyl, n-butyl, i-butyl, t-butyl and 3-methyl-butyl. Examples of C₁-C₄ alkyl groups and moieties include methyl, ethyl, n-propyl, i-propyl, n-butyl, i-butyl and t-butyl. For the avoidance of doubt, where two alkyl moieties are present in a group, the alkyl moieties may be the same or different.

As used herein, a C₁-C₄ alkylene group or moiety is a linear or branched alkylene group or moiety. Examples include methylene, ethylene and n-propylene groups and moieties.

Typically, as used herein, a C₆-C₁₀ aryl group or moiety is phenyl or naphthyl. Phenyl is preferred.

As used herein, a halogen is typically chlorine, fluorine, bromine or iodine and is preferably chlorine, bromine or fluorine.

As used herein, a C₁-C₄ alkoxy group is typically a said C₁-C₄ alkyl group attached to an oxygen atom. A haloalkyl or haloalkoxy group is typically a said alkyl or alkoxy group substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom, for example chlorine and fluorine. Particularly preferred haloalkyl groups are —CF₃ and —CCl₃. Particularly preferred haloalkoxy groups are —OCF₃ and —OCCl₃.

As used herein a C₁-C₄ hydroxyalkyl group is a C₁-C₄ alkyl group substituted by one or more hydroxy groups. Typically, it is substituted by one, two or three hydroxy groups. Preferably, it is substituted by a single hydroxy group. A preferred hydroxyalkyl group is —CH₂—OH.

As used herein, a C₁-C₄ thioalkyl group is a C₁-C₄ alkyl group substituted by one or more thio groups (—SH). Typically, it is substituted by one, two or three thio groups. Preferably, it is substituted by a single thio group.

As used herein, C₁-C₄ aminoalkyl group is a C₁-C₄ alkyl group substituted by one or more —NH₂ groups. Typically, it is substituted by one, two or three —NH₂ groups. Preferably, it is substituted by a single —NH₂ group.

As used herein, a 5- to 10-membered heteroaryl group or moiety is a monocyclic 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl, pyridazolyl and pyrazolyl groups. Preferred examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl and pyrazolyl groups. Furanyl, thienyl, pyridazolyl, pyrazolyl, pyrimidinyl and thiazolyl groups are preferred. Furanyl, thienyl, pyrimidinyl and thiazolyl groups are further preferred. Furanyl, thienyl and thiazolyl groups are still further preferred.

As used herein, a 5- to 10-membered heterocyclyl group or moiety is a monocyclic non-aromatic, saturated or unsaturated C₅-C₁₀ carbocyclic ring in which one or more, for example 1, 2 or 3, of the carbon atoms are replaced with a moiety selected from N, O, S, S(O) and S(O)₂. Typically, it is a 5- to 6-membered ring.

Suitable heterocyclyl groups and moieties include pyrazolidinyl, piperidyl, piperazinyl, thiomorpholinyl, S-oxo-thiomorpholinyl, S,S-dioxo-thiomorpholinyl, morpholinyl, pyrrolidinyl, pyrrolinyl, imidazolidinyl, imidazolinyl, 1,3-dioxolanyl, 1,4-dioxolyl and pyrazolinyl groups and moieties. Piperazinyl, thiomorpholinyl, S,S-dioxothiomorpholinyl, morpholinyl and 1,3-dioxolanyl groups and moieties are preferred.

Typically, the aryl, heteroaryl and heterocyclyl moieties in the R₁ substituent are unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, cyano, —COR′, —CO₂R′, —S(O)R′, —S(O)₂R′ and -L′-X-L″-Y substituents, wherein R′, L′, X, L″ and Y are as defined above.

Typically, L′ is a direct bond or a C₁₋₂ alkylene group. Typically, X is —O— or —NR′— wherein R′ is as defined above. Typically, L″ is a direct bond or a C₁-C₂ alkylene group, preferably a C₁-C₂ alkylene group. Typically, Y is hydrogen, —COR′, —CO₂R′, —S(O)₂R′, or —S(O)R′, wherein R′ is a C₁-C₄ alkyl group. Preferably, Y is hydrogen, —COR′, —S(O)₂R′ or —S(O)R′, wherein R′ is a C₁-C₄ alkyl group.

Preferably, the aryl, heteroaryl and heterocyclyl moieties in the R₁ substituent are unsubstituted or substituted with 1 or 2 substituents selected from halogen, C₁-C₂ alkyl, C₁-C₂ haloalkyl, C₁-C₂ hydroxyalkyl, cyano, —COR′, —CO₂R′, —S(O)R′, —S(O)₂R′, —(C₁-C₂ alkyl)-NR′R″, C₁-C₂ alkoxy, —NR′—COR′, NR′—CO₂R′, —(C₁-C₂ alkyl)-NR′—CO₂R′, —NR′—S(O)₂—R′ and —C₁-C₂ alkyl)-NR′—(C₁-C₂ alkyl)-S(O)₂—R″ substituents, wherein each R′, R″ and R′ are the same or different and represent hydrogen or C₁-C₂ alkyl.

In one embodiment the quinazoline derivative of formula (Ia) is a quinazoline derivative of formula (I) and, typically, the aryl, heteroaryl and heterocyclyl moieties in the R₁ substituent are unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, —COR′, —CO₂R′, —S(O)R′, —S(O)₂R′ and -L′-X-L″-Y substituents, wherein R′, L′, X, L″ and Y are as defined above. Preferably, the aryl, heteroaryl and heterocyclyl moieties in the R₁ substituent are unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, —COR′ and -L′-X-L″-Y substituents, wherein R′, L′, X, L″ and Y are as defined above. More preferably, the aryl, heteroaryl and heterocyclyl moieties in the R₁ substituent are unsubstituted or substituted with 1 or 2 substituents selected from halogen, C₁-C₂ alkyl, C₁-C₂ alkoxy, C₁-C₂ haloalkyl, C₁-C₂ hydroxyalkyl, —COR′, —CO₂R′, —S(O)R′, —S(O)₂R′, —(C₁-C₂ alkyl)-NR′R″ and —(C₁-C₂ alkyl)-NR′—(C₁-C₂ alkyl)-S(O)₂—R″ substituents, wherein each R′ and R″ are the same or different and represent hydrogen or C₁-C₂ alkyl. Yet more preferably, the aryl, heteroaryl and heterocyclyl moieties in the R₁ substituent are unsubstituted or substituted with 1 or 2 substituents selected from halogen, C₁-C₂ alkyl, C₁-C₂ haloalkyl, C₁-C₂ hydroxyalkyl, —COR′, —(C₁-C₂ alkyl)-NR′R″ and —(C₁-C₂ alkyl)-NR′—(C₁-C₂ alkyl)-S(O)₂—R″ substituents, wherein each R′ and R″ are the same or different and represent hydrogen or C₁-C₂ alkyl.

Typically, A is a phenyl, 5- to 6-membered heteroaryl or 5- to 6-membered heterocyclyl group. Preferably, A is a phenyl or 5- to 6-membered heteroaryl group. More preferably, A is a phenyl, furanyl, thienyl, pyrimidinyl, thiazolyl or pyridazolyl group. Yet more preferably, A is a phenyl, furanyl, thienyl, pyrimidinyl or thiazolyl group. Most preferably, A is a phenyl, furanyl, thienyl or thiazolyl group.

Typically, L is a direct bond or a C₁-C₂ alkylene group.

Typically, A′ is a 5- to 6-membered heteroaryl or 5- to 6-membered heterocyclyl group. Preferably, when A′ is a 5- to 6-membered heteroaryl group it is a pyrazolyl group.

More preferably, A′ is a 5- to 6-membered heterocyclyl or heteroaryl group, which group is unsubstituted or substituted with 1, 2 or 3 substituents selected from halogen, C₁-C₄ alkyl, C₁-C₄ haloalkyl and C₁-C₄ haloalkoxy substituents. Most preferably, A′ is a morpholinyl, thiomorpholinyl, piperazinyl, 1,3-dioxolanyl, S,S-dioxothiomorpholino or pyrazolyl group which is unsubstituted or substituted by one or two substituents selected from C₁-C₂ alkyl, halogen and C₁-C₂ haloalkyl groups.

In one embodiment the quinazoline derivative of formula (Ia) is a quinazoline derivative of formula (I) and, preferably A′ is a 5- to 6-membered heterocyclyl group. More preferably, A′ is a 5- to 6-membered heterocyclyl group which is unsubstituted or substituted with 1, 2 or 3 substituents selected from halogen, C₁-C₄ alkyl, C₁-C₄ haloalkyl and C₁-C₄ haloalkoxy substituents. Most preferably, A′ is a morpholinyl, thiomorpholinyl, piperazinyl, 1,3-dioxoanyl or S,S-dioxothiomorpholino group which is unsubstituted or substituted by one or two substituents selected from C₁-C₂ alkyl, halogen and C₁-C₂ haloalkyl groups.

Typically, Z is —O—, —CONR′—, —NR′C(O)— or —NR′CO₂—, wherein R′ is as defined above. Preferably Z is —O—, —CONH—, —CON(C₁-C₂ alkyl)-, —NHC(O)— or —NHCO₂—.

Typically R₁ is halogen, C₁-C₄ alkyl, C₁-C₄ haloalkyl, C₁-C₄ alkoxy, C₁-C₄ haloalkoxy, —CO₂R′, —CONR′R″, -A, -A-L-A′, -Z-L-A, or -A-L-Z-L-A wherein R′, R″, A, L, A′ and Z are as defined above. Preferably, R₁ is halogen, C₁-C₂ alkoxy, C₁-C₂ haloalkoxy, —CONR′R″, -A, —Ar-L-A′, -Z-L-A or —Ar-Z-L-Ar, wherein R′ and R″ are the same or different and each represent hydrogen or a C₁-C₂ alkyl group, A and A′ are as defined above, Ar is an unsubstituted furanyl or unsubstituted phenyl group, L is a direct bond or a methylene group and Z is —O—, —C(O)NR′—, —NR′C(O)— or —NR′CO₂—, wherein R′ is as defined above.

In one embodiment the quinazoline derivative of formula (Ia) is a quinazoline derivative of formula (I) and, typically, R₁ is halogen, C₁-C₄ alkyl, C₁-C₄ haloalkyl, C₁-C₄ alkoxy, C₁-C₄ haloalkoxy, -A or -A-L-A′, wherein A, L and A′ are as defined above. Preferably, R₁ is halogen, C₁-C₂ alkoxy, C₁-C₂ haloalkoxy, -A or —Ar-L-A′ wherein A and A′ are as defined above, Ar is an unsubstituted furanyl group and L is a direct bond or a methylene group.

Typically, R₂ is hydrogen, C₁-C₄ alkyl or C₁-C₄ alkoxy. Preferably, R₂ is hydrogen or C₁-C₂ alkoxy.

Typically, R₃ is hydrogen, C₁-C₂ alkyl, C₁-C₂ haloalkyl or C₁-C₂ alkoxy. Preferably, R₃ is hydrogen, methyl, trifluoromethyl or methoxy.

Typically, R₄ is hydrogen or C₁-C₆ alkyl.

Preferred compounds of the invention are those in which:
R₁ is halogen, C₁-C₄ alkyl, C₁-C₄ haloalkyl, C₁-C₄ alkoxy, C₁-C₄ haloalkoxy, —CO₂R′, —CONR′R″, -A, -A-L-A′, -Z-L-A, or -A-L-Z-L-A;
R₂ is hydrogen, C₁-C₄ alkyl or C₁-C₄ alkoxy;
R₃ is hydrogen, C₁-C₂ alkyl, C₁-C₂ haloalkyl or C₁-C₂ alkoxy;
R₄ is hydrogen or C₁-C₆ alkyl;
A is a phenyl, 5- to 6-membered heteroaryl or 5- to 6-membered heterocyclyl group;
L is a direct bond or a C₁-C₂ alkylene group;
A′ is a 5- to 6-membered heteroaryl or heterocyclyl group; and
Z is —O—, —CONR′—, —NR′C(O)— or —NR′CO₂—, wherein R′ is hydrogen or C₁-C₄ alkyl,

the aryl, heteroaryl and heterocyclyl moieties in the R₁ substituent being unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, cyano, —COR′, —CO₂R′, —S(O)R′, —S(O)₂R′ and -L′-X-L″-Y substituents, wherein R′, L′, X, L″ and Y are as defined above.

Further preferred compounds of the invention are those in which the quinazoline derivative of formula (Ia) is a quinazoline derivative of formula (I), wherein:
R₁ is halogen, C₁-C₄ alkyl, C₁-C₄ haloalkyl, C₁-C₄ alkoxy, C₁-C₄ haloalkoxy, -A or -A-L-A′;
R₂ is hydrogen, C₁-C₄ alkyl or C₁-C₄ alkoxy;
A is a phenyl, 5- to 6-membered heteroaryl or 5- to 6-membered heterocyclyl group;
L is a direct bond or a C₁-C₄ alkylene group; and
A′ is a 5- to 6-membered heteroaryl or heterocyclyl group,

the aryl, heteroaryl and heterocyclyl moieties in the R₁ substituent being unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, —COR′, —CO₂R′, —S(O)R′, —S(O)₂R′ and -L′-X-L″-Y substituents, wherein R′, L′, X, L″ and Y are as defined above.

Further preferred compounds of the invention are those in which the quinazoline derivative of formula (Ia) is a quinazoline derivative of formula (I), wherein:
R₁ is halogen, C₁-C₄ alkyl, C₁-C₄ haloalkyl, C₁-C₄ alkoxy, C₁-C₄ haloalkoxy, -A or -A-L-A′;
R₂ is hydrogen, C₁-C₄ alkyl or C₁-C₄ alkoxy;
A is a phenyl, 5- to 6-membered heteroaryl or 5- to 6-membered heterocyclyl group;
L is a direct bond or a C₁-C₄ alkylene group; and
A′ is a 5- to 6-membered heteroaryl or heterocyclyl group, the aryl, heteroaryl and heterocyclyl moieties in the R₁ substituent being unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, —COR′ and -L′-X-L″-Y substituents, wherein R′, L′, X, L″ and Y are as defined above.

Further preferred compounds of the invention are those wherein:
R₁ is halogen, C₁-C₂ alkoxy, C₁-C₂ haloalkoxy, —CONR′R″, -A, —Ar-L-A′, -Z-L-A, or —Ar-Z-L-Ar, wherein R′ and R″ are the same or different and each represent hydrogen or a C₁-C₂ alkyl group;
R₂ is hydrogen or C₁-C₂ alkoxy;
A is a phenyl or 5- to 6-membered heteroaryl group, for example furanyl, thienyl, pyrimidinyl and thiazolyl, which group is unsubstituted or substituted with 1 or 2 substituents selected from halogen, C₁-C₂ alkyl, C₁-C₂ haloalkyl, C₁-C₂ hydroxyalkyl, cyano, —COR′, —CO₂R′, —S(O)R′, —S(O)₂R′, —(C₁-C₂ alkyl)-NR′R″, C₁-C₂ alkoxy, —NR′—COR′, NR′—CO₂R′, —(C₁-C₂ alkyl)-NR′—CO₂R′, —NR′—S(O)₂—R′ and —(C₁-C₂ alkyl)-NR′—(C₁-C₂ alkyl)-S(O)₂—R″ substituents, wherein each R′ and R″ are the same or different and represent hydrogen or C₁-C₂ alkyl;
Ar is an unsubstituted furanyl or unsubstituted phenyl group;
L is a direct bond or a methylene group;
A′ is a 5- to 6-membered heterocyclyl or heteroaryl group, for example morpholinyl, thiomorpholinyl, piperazinyl, 1,3-dioxolanyl, S,S-dioxothiomorpholinyl and pyrazolyl, which is unsubstituted or substituted by one or two substituents selected from C₁-C₂ alkyl, halogen and C₁-C₂ haloalkyl groups; and
Z is —O—, —CONH—, —CON(C₁-C₂ alkyl)- or —NHC(O)—.

Further preferred compounds of the invention are those in which the quinazoline derivative of formula (Ia) is a quinazoline derivative of formula (I), wherein:
R₁ is halogen, C₁-C₂ alkoxy, C₁-C₂ haloalkoxy, -A or —Ar-L-A′;
R₂ is hydrogen or C₁-C₂ alkoxy;
A is a phenyl or 5- to 6-membered heteroaryl group, for example furanyl, thienyl, pyrimidinyl and thiazolyl, which group is unsubstituted or substituted with 1 or 2 substituents selected from halogen, C₁-C₂ alkyl, C₁-C₂ alkoxy, C₁-C₂ haloalkyl, C₁-C₂ hydroxyalkyl, —COR′, —CO₂R′, —S(O)R′, —S(O)₂R′, —(C₁-C₂ alkyl)NR′R″ and —(C₁-C₂ alkyl)-NR′—(C₁-C₂ alkyl)-S(O)₂—R″ substituents, wherein each R′ and R″ are the same or different and represent hydrogen or C₁-C₂ alkyl;
Ar is an unsubstituted furanyl group;
L is a direct bond or a methylene group; and
A′ is a 5- to 6-membered heterocyclyl group, for example morpholinyl, thiomorpholinyl, piperazinyl, 1,3-dioxoanyl and S,S-dioxothiomorpholinyl, which is unsubstituted or substituted by one or two substituents selected from C₁-C₂ alkyl, halogen and C₁-C₂ haloalkyl groups.

Further preferred compounds of the invention are those in which the quinazoline derivative of formula (Ia) is a quinazoline derivative of formula (I), wherein:
R₁ is halogen, C₁-C₂ alkoxy, C₁-C₂ haloalkoxy, -A or —Ar-L-A′;
R₂ is hydrogen or C₁-C₂ alkoxy;
A is a phenyl or 5- to 6-membered heteroaryl group, for example furanyl, thienyl and thiazolyl, which group is unsubstituted or substituted with 1 or 2 substituents selected from halogen, C₁-C₂ alkyl, C₁-C₂ haloalkyl, C₁-C₂ hydroxyalkyl, —COR′, —(C₁-C₂ alkyl)-NR′R″ and —(C₁-C₂ alkyl)-NR′—(C₁-C₂ alkyl)-S(O)₂—R″ substituents, wherein each R′ and R″ are the same or different and represent hydrogen or C₁-C₂ alkyl;
Ar is an unsubstituted furanyl group;
L is a direct bond or a methylene group; and
A′ is a 5- to 6-membered heterocyclyl group, for example morpholinyl, thiomorpholinyl, piperazinyl, 1,3-dioxoanyl and S,S-dioxothiomorpholinyl, which is unsubstituted or substituted by one or two substituents selected from C₁-C₂ alkyl, halogen and C₁-C₂ haloalkyl groups.

Particularly preferred compounds of formula (Ia) include:
(6-bromo-quinazolin-4-yl)-(4-morpholin-4-yl-phenyl)-amine;
(6-iodo-quinazolin-4-yl)-(4-morpholin-4-yl-phenyl)-amine;
(6,7-dimethoxy-quinazolin-4-yl)-(4-morpholin-4-yl-phenyl)-amine;
(6-trifluoromethoxy-quinazolin-4-yl)-(4-morpholin-4-yl-phenyl)-amine;
(6-furan-2-yl-quinazolin-4-yl)-(4-morpholin-4-yl-phenyl)-amine;
[6-(5-[1,3]dioxolan-2-yl-furan-2-yl)-quinazolin-4-yl]-(4-morpholin-4-yl-phenyl)-amine;
5-[4-(4-morpholin-4-yl-phenylamino)-quinazolin-6-yl]-furan-2-carbaldehyde;
{5-[4-(4-morpholin-4-yl-phenylamino)-quinazolin-6-yl]-furan-2-yl}-methanol;
(6-{5-[(2-methanesulfonyl-ethylamino)-methyl]-furan-2-yl}-quinazolin-4-yl)-(4-morpholin-4-yl-phenyl)-amine;
{6-[5-(1,1-dioxo-1-λ-6-thiomorpholin-4-ylmethyl)-furan-2-yl]-quinazolin-4-yl}-(4-morpholin-4-yl-phenyl)-amine;
{6-[5-(4-methyl-piperazin-1-ylmethyl)-furan-2-yl]-quinazolin-4-yl}-(4-morpholin-4-yl-phenyl)-amine;
[6-(5-morpholin-4-ylmethyl-furan-2-yl)-quinazolin-4-yl]-(4-morpholin-4-yl-phenyl)-amine;
[6-(5-dimethylaminomethyl-furan-2-yl)-quinazolin-4-yl]-(4-morpholin-4-yl-phenyl)-amine;
[6-(5-methylaminomethyl-furan-2-yl)-quinazolin-4-yl]-4-morpholin-4-yl-phenyl)-amine;
(4-morpholin-4-yl-phenyl)-(6-thiophen-2-yl-quinazolin-4-yl)-amine;
(6-chloro-quinazolin-4-yl)-(4-morpholin-4-yl-phenyl)-amine;
(4-morpholin-4-yl-phenyl)-(6-o-tolyl-quinazolinyl)-amine;
(4-morpholin-4-yl-phenyl)-(6-thiazol-2-yl-quinazolin-4-yl)-amine;
(4-morpholin-4-yl-phenyl)-[6-(3-pyrazol-1-yl-phenyl)-quinazolin-4-yl]-amine;
4-[4-(4-morpholin-4-yl-phenylamino)-quinazolin-6-yl]-benzonitrile;
[6-(2-methoxy-pyrimidin-5-yl)-quinazolin-4-yl]-(4-morpholin-4-yl-phenyl)-amine;
[6-(4-methyl-thiophen-2-yl)quinazolin-4-yl]-4-morpholin-4-yl-phenyl)-amine;
5-[4-(4-morpholin-4-yl-phenylamino)-quinazolin-6-yl]-thiophene-2-carboxylic acid;
[6-(4-methanesulfonyl-phenyl)-quinazolin-4-yl]-(4-morpholin-4-yl-phenyl)-amine;
furan-2-carboxylic acid [4-(4-morpholin-4-yl-phenylamino)-quinazolin-6-yl]-amide;
4-(4-morpholin-4-yl-phenylamino)-quinazoline-6-carboxylic acid 4-methoxy-benzylamide;
3-{[4-(4-morpholin-4-yl-phenylamino)-quinazoline-6-carbonyl]amino}-benzoic acid ethyl ester;
4-(4-morpholin-4-yl-phenylamino)-quinazoline-6-carboxylic acid 3-methoxy-benzylamide;
4-(4-morpholin-4-yl-phenylamino)-quinazoline-6-carboxylic acid 4-methyl-benzylamide;
4-(4-morpholin-4-yl-phenylamino)-quinazoline-6-carboxylic acid methylamide;
4-(4-morpholin-4-yl-phenylamino)-quinazoline-6-carboxylic acid dimethylamide;
4-(4-morpholin-4-yl-phenylamino)-quinazoline-6-carboxylic acid ethylamide;
N-{3-[4-(4-morpholin-4-yl-phenylamino)-quinazolin-6-yl]-phenyl}-acetamide;
{4-[4-(4-morpholin-4-yl-phenylamino)quinazolin-6-yl]-phenyl}-carbamic acid benzyl ester;
N-{4-[4-(4-morpholin-4-yl-phenylamino)-quinazolin-6-yl]-phenyl}acetamide;
{4-[4-(4-morpholin-4-yl-phenylamino)-quinazolin-6-yl]-phenyl}-carbamic acid tert-butyl ester;
{3-[4-(4-morpholin-4-yl-phenylamino)-quinazolin-6-yl]benzyl}-carbamic acid tert-butyl ester;
N-{3-[4-(4-morpholin-4-yl-phenylamino-quinazolin-6-yl]-phenyl}-methanesulfonamide;
(6-iodo-quinazolin-4-yl)-(4-morpholin-4-yl-2-trifluoromethyl-phenyl)-amine;
(4-morpholin-4-yl-2-trifluoromethyl-phenyl)-(6-thiophen-2-yl-quinazolin-4-yl)-amine;
(6-iodo-quinazolin-4-yl)-(3-methoxy-4-morpholin-4-yl-phenyl)-amine;
(3-methoxy-4-morpholin-4-yl-phenyl)-(6-thiophen-2-yl-quinazolin-4-yl)-amine;
(2-methyl-4-morpholin-4-yl-phenyl)-(6-thiophen-2-yl-quinazolin-4-yl)-amine;
(3-methyl-4-morpholin-4-yl-phenyl-(6-thiophen-2-yl-quinazolin-4-yl)-amine;
ethyl-(4-morpholin-4-yl-phenyl)-(6-thiophen-2-yl-quinazolin-4-yl)-amine;
(6-iodo-quinazolin-4-yl)-methyl-(4-morpholin-4-yl-phenyl)-amine;
(6-iodo-quinazolin-4-yl)-(3-methyl-butyl)-(4-morpholin-4-yl-phenyl)-amine;
isopropyl-(4-morpholin-4-yl-phenyl)-(6-thiophen-2-yl-quinazolin-4-yl)-amine;
(3-methyl-butyl-(4-morpholin-4-yl-phenyl)-6-thiophen-2-yl-quinazolin-4-yl)-amine;
[6-(2-benzyloxy-phenyl)-quinazolin-4-yl]-(4-morpholin-4-yl-phenyl)-amine;
[6-(4-benzyloxy-phenyl)-quinazolin-4-yl]-(4-morpholin-4-yl-phenyl)-amine; and pharmaceutically acceptable salts thereof.

Compounds of formula (Ia) containing one or more chiral centre may be used in enantiomerically or diastereoisomerically pure form, or in the form of a mixture of isomers. For the avoidance of doubt, the compounds of formula (Ia) can, if desired, be used in the form of solvates. Further, for the avoidance of doubt, the compounds of the invention may be used in any tautomeric form.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

The compounds of the invention in which R₁ is other than hydrogen or halogen, can, for example, be prepared according to the following reaction schemes.

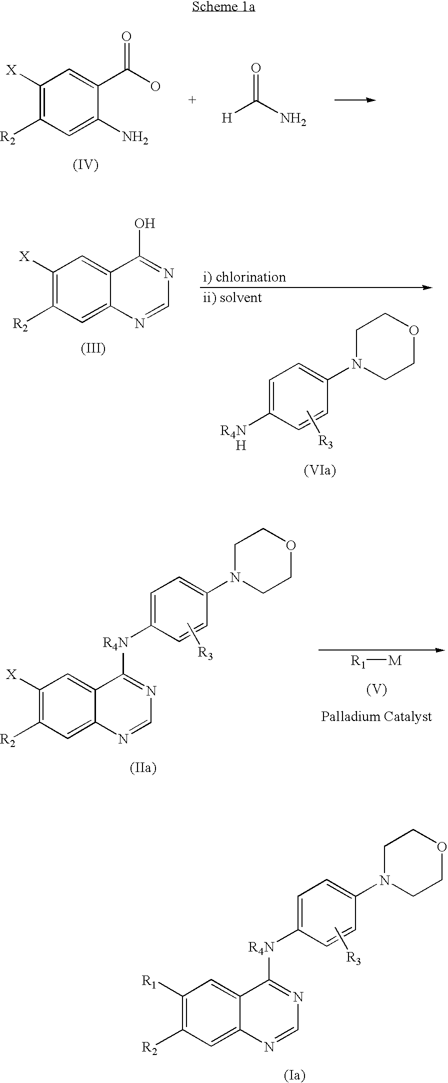

In one embodiment, Scheme 1a is represented by Scheme 1.

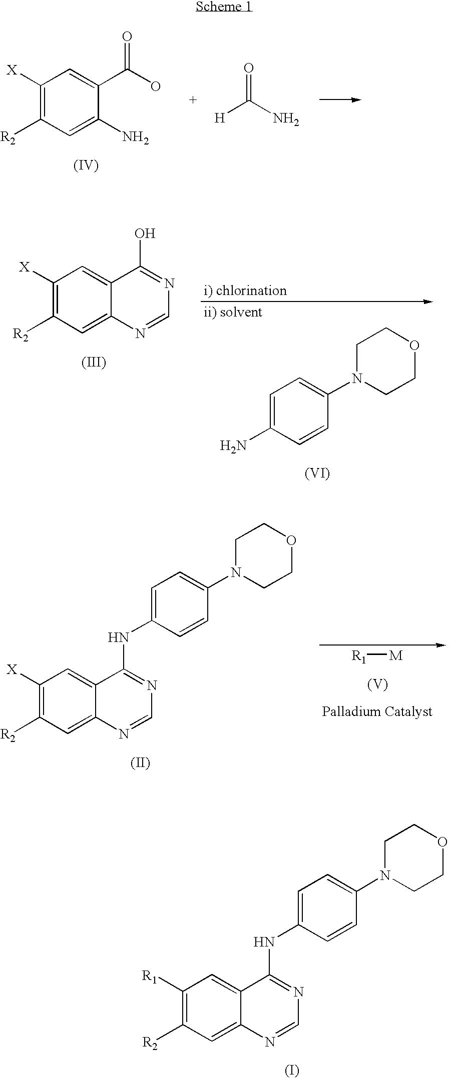

As will be evident to one of skill in the art, X in the above reaction schemes is an appropriate leaving group, for example halogen.

Referring to Schemes 1a and 1, the treatment of compounds of formula (IIa) and (II), respectively, with an organometallic reagent (V) is conveniently carried out in a suitable solvent (such as tetrahydrofuran, dimethylformamide or toluene) and at elevated temperature (eg from 50° C. to reflux). Conveniently, the reaction is performed under palladium catalysis (eg 20 mol % tris (dibenzylideneacetone)dipalladium (II) or 20 mol % dichlorobis (triphenylphosphine)palladium (0)) in the presence of an organic base (eg triethylamine) or an inorganic base (eg sodium carbonate or potassium phosphate). Where reagent (V) is an organostannane (eg M=SnBu₃), one skilled in the art will recognise the reaction as an example of a Stille coupling where additional additives may be beneficial eg lithium chloride, silver oxide and conveniently the reaction is performed in toluene and at reflux temperature. Where reagent (V) is a boronic acid derivative, one skilled in the art will recognise the reaction as an example of a Suzuki-Miyaura coupling which may be conveniently performed at 60° C. in tetrahydrofuran.

Referring to Schemes 1a and 1, the conversion of compounds of formula (III) to compounds of formula (IIa) and (II), respectively, is accomplished by converting the 4-hydroxy group of compounds of formula (III) to a suitable leaving group eg chloro using a reagent such as thionyl chloride as solvent with the addition of a catalytic activator eg dimethylformamide, and subsequent reaction with 4-morpholinoaniline in a suitable solvent eg acetonitrile.

Referring to Schemes Ia and I, the conversion of compounds of formula (IV) to compounds of formula (III) will be well known to one skilled in the art, being conveniently performed with formamide as solvent and at elevated temperature eg reflux.

Compounds of formula (Ia) or (I) in which R₁ is hydrogen or halogen can, of course, be prepared using compounds of formula (IV) in which X is hydrogen or halogen, converting the compounds of formula (IV) to corresponding compounds of formula (III) as described above and reacting with a compound of formula (VIa) or (VI), respectively, as described above. Compounds of formula (Ia) or (I) in which R₁ is alkyl, haloalkyl, alkoxy and haloalkoxy can, of course, also be prepared in an analogous manner.

The starting materials in the above reaction scheme are known compounds, or can be prepared by analogy with known methods. In particular, compounds of formula (VIa) may be prepared by known methods such as those outlined in scheme 2.

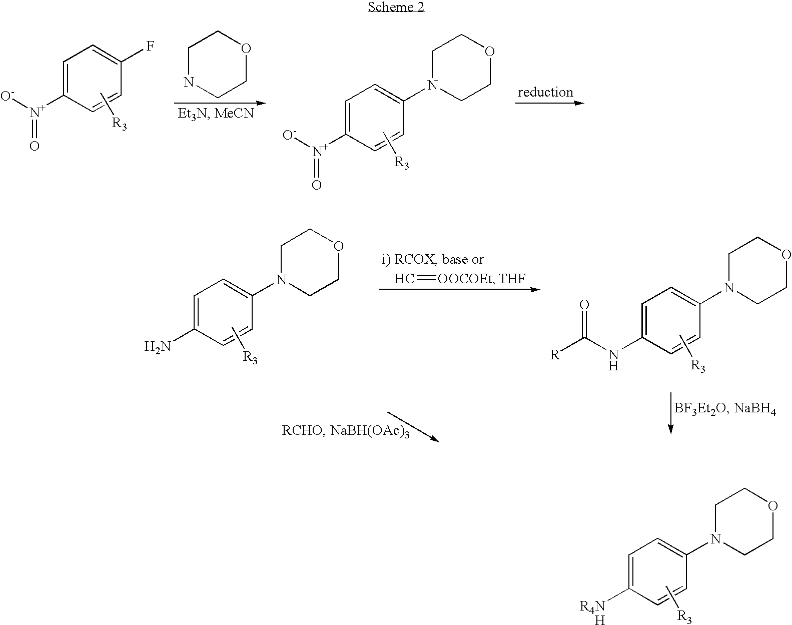

The compounds of the present invention are therapeutically useful. The present invention therefore provides a quinazoline derivative of the formula (Ia), as defined above, or a pharmaceutically acceptable salt thereof, for use in treating the human or animal body. Also provided is a pharmaceutical composition comprising a quinazoline derivative of the formula (Ia), as defined above, or a pharmaceutically acceptable salt thereof, and a pharmaceutically acceptable carrier or diluent.

Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

As explained above, the compounds of the invention are active against a flaviviridae infection. The present invention therefore provides the use of a quinazoline derivative of the formula (Ia), as defined above, or a pharmaceutically acceptable salt thereof, in the manufacture of a medicament for use in treating or preventing a flaviviridae infection. Also provided is a method for treating a patient suffering from or susceptible to a flaviviridae infection, which method comprises administering to said patient an effective amount of a quinazoline derivative of formula (Ia) or a pharmaceutically acceptable salt thereof.

The flaviviridae family contains three genera. These are hepacivirus, flavivirus and pestivirus. The compounds of the invention are active in treating or preventing a hepacivirus infection, a flavivirus infection or a pestivirus infection.

Typical pestivirus infections which can be treated with the compounds of the invention include bovine viral diarrhea virus, classical swine fever virus and border disease virus.

Typical flavivirus infections which can be treated with the compounds of the invention include yellow fever virus, dengue fever virus, Japanese encephalitis virus and tick borne encephalitis virus.

Typical hepacivirus infections that can be treated with the compounds of the invention include hepatitis C virus.

Compounds of the present invention are especially active against hepatitis C. Typically, said flavivirus is therefore hepatitis C virus.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

Compounds of the present invention may be used in conjunction with known anti-viral agents. Preferred known anti-viral agents in this regard are interferon and ribavirin, and derivatives thereof, which are known for the treatment of hepatitis C (Clinical Microbiology Reviews, January 2000, 67-82). The said medicament therefore typically further comprises interferon or a derivative thereof and/or ribavirin or a derivative thereof. Further, the present invention provides a pharmaceutical composition comprising:
(a) a quinazoline derivative of the formula (Ia), as defined above, or a pharmaceutically acceptable salt thereof;
(b) interferon or a derivative thereof and/or ribavirin or a derivative thereof; and
(c) a pharmaceutically acceptable carrier or diluent.

Also provided is a product comprising:
(a) a quinazoline derivative of the formula (Ia), as defined above, or a pharmaceutically acceptable salt thereof; and
(b) interferon or a derivative thereof and/or ribavirin or a derivative thereof,

for separate, simultaneous or sequential use in the treatment of the human or animal body.

A preferred interferon derivative is PEG-interferon. A preferred ribavirin derivative is viramidine.

A therapeutically effective amount of a compound of the invention is administered to a patient. A typical dose is from about 0.01 to 100 mg per kg of body weight, according to the activity of the specific compound, the age, weight and conditions of the subject to be treated, the type and severity of the disease and the frequency and route of administration. Preferably, daily dosage levels are from 0.05 to 16 mg per kg of body weight, more preferably, from 0.05 to 1.25 mg per kg of body weight.

The following Examples illustrate the invention. They do not however, limit the invention in any way. In this regard, it is important to understand that the particular assay used in the Examples section is designed only to provide an indication of anti-viral activity. There are many assays available to determine such activity, and a negative result in any one particular assay is therefore not determinative.

### EXAMPLES

All temperatures are in ° C. Thin layer chromatography (TLC) was carried out on Si 60G coated plastic plates with uv254 indicator (Polygram). All NMR spectra were obtained at 250 MHz in d⁶-DMSO unless stated otherwise.

### LC-MS Conditions

Samples were run on a MicroMass ZMD, using electrospray with simultaneous positive-negative ion detection.

### Column: Synergi Hydro-RP, 30×4.6 mm I.D, 4 μm.

Gradient: 95:5 to 5:95 v/v H₂O/CH₃CN+0.05% Formic Acid over 4.0 min, hold 3 min, return to 95:5 v/v H₂O/CH₃CN+0.05% Formic Acid over 0.2 min and hold at 95:5 v/v H₂O/CH₃CN+0.05% Formic Acid over 3 min.

### Detection: PDA 250-340 nm.

Flow rate: 1.5 ml/min

### Example 1

### (6-Bromo-quinazolin-4-yl)-(4-morpholin-4-yl-phenyl)-amine

5-Bromo-2-aminobenzoic acid (5 g, 23.1 mmol) was suspended in formamide (5 eq) and heated to 155° C. under N₂ for 16 h. The mixture was allowed to cool and added to water. The resulting precipitate was isolated by filtration and dried to give an intermediate 6-bromoquinazolin-4-ol. A portion of this material (1 g) was dissolved in thionyl chloride (10 ml) and DMF (0.3 ml) added before being refluxed for 5 h. The solvents were removed and the residue azeotroped with toluene (3×10 ml) to remove traces of thionyl chloride. The resulting material was dissolved in MeCN (10 ml), 4-morpholinoaniline (1.1 eq, Lancaster) added and the reaction mixture heated to reflux for 24 hr. On cooling the precipitate was isolated by filtration to give the title compound as a beige solid (867 mg).

δ(DMSO) 11.5 (1H br s); 9.17 (1H, d, J2.5 Hz); 8.9 (1H, s); 8.22 (1H, dd, J 8.8, 1.9 Hz); 7.88 (1H, d, J 8.8 Hz); 7.61 (2H, d, J 8.8 Hz); 7.06 (2H, d, J 8.8 Hz); 3.78 (4H, m); 3.18 (4H, m)

LC-MS ES+=385, rt 3.89

### Example 2

### (6-Iodo-quinazolin-4-yl)-(4-morpholin-4-yl-phenyl)-amine

By a similar procedure to Example 1, using 5-iodo-2-aminobenzoic acid as starting material gave the title compound as an orange solid (568 mg)

δ (DMSO) 11.5 (1H, br s); 9.17 (1H, d, J2.5 Hz); 8.89 (1H, s); 8.22 (1H, dd, J 8.8, 1.9 Hz); 7.88 (1H, d, J 8.8 Hz); 7.62 (2H, d, J 8.8 Hz); 7.07 (2H, d, J 8.8 Hz); 3.78 (4H, m); 3.18 4H, m)

LC-MS ES+=433, rt 3.97

### Example 3

### (6,7-Dimethoxy-quinazolin-4-yl)-(4-morpholinyl-phenyl)-amine

By a similar procedure to Example 1, using 4,5-dimethoxy-2-aminobenzoic acid as starting material gave the title compound.

LC-MS ES+=367 rt 3.76

### Example 4

### (6-trifluoromethoxy-quinazolinyl-4-yl)-(4-morpholin-4-yl-phenyl)-amine

By a similar procedure to Example 1, using 5-trifluoromethoxy-2-amino benzoic acid as starting material, the title compound was obtained (110 mg)

δ (DMSO) 10.0 (1H, s); 8.66 (1H, s); 8.62 (1H, s); 7.91 (2H, s); 7.68 (2H, d, J 8.85 Hz); 7.0 (2H, d, J 8.85 Hz); 3.80 (2H, m); 3.10 (2H, m)

LC-MS 391 rt. 4.89

### Example 5

### (6-Furan-2-yl-quinazolin-4-yl)-(4-morpholin-4-yl-phenyl)-amine

(6-Iodo-quinazoline-4-yl)-(4-morpholin-4-yl-phenyl)-amine (Example 2, 0.23 mmol) was dissolved in toluene (5 ml) and treated with tributylstannylfuran (Aldrich, 1.1 eq), lithium chloride (5 eq) and bis(triphenylphosphine) palladium dichloride (5 mol %) and heated to reflux under nitrogen for 24 h. Aqueous workup followed by column chromatography gave the title compound (22 mg)

δ (DMSO) 9.92 (1H, s); 8.89 (1H, s); 8.55 (1H, s); 8.23 (1H, dd, J 8.85, 1.89 Hz); 7.95 (1H, d, 1.26 Hz); 7.84 (1H, d, 8.85 Hz); 7.72 (2H, d, J 8.85 Hz); 7.19 (1H, d, J 1.89 Hz); 7.07 (2H, d, 9.48 Hz); 6.77 (1 h, dd, J 3.16 Hz, 1.89 Hz); 3.83 (4H, m); 3.18 (4H, m)

LC-MS ES+=373 rt 4.31

### Example 6

### [6-(5-[1,3]Dioxolan-2-yl-furan-2-yl)quinazolin-4-yl]-(4-morpholin-4-yl-phenyl)-amine

The method of Example 5, using 5-[1,3]dioxolan-2-yl-2-tributylstannylfuran gave the title compound (30 mg)

δ (DMSO) 8.56 (1H, s); 8.24 (1H, s); 7.96 (1H, dd, J 8.85, 1.89 Hz); 7.86 (1H, d, J 8.85 Hz); 7.75 (2H, d, 8.85 Hz); 6.88 (2H, d, J 8.85 Hz); 6.83 (1H, d, J 3.16 Hz); 6.5 (1H, d, J 3.8 Hz); 5.95 (1H, s); 4.11 (2H, m); 4.01 (2H, m); 3.81 (4H, m); 3.11 (4H, m)LC-MS ES+=445 rt 4.21

### Example 7

### 5-[4-(4-Morpholin 4-yl-phenylamino)-quinazolin-6-yl]-furan-2-carbaldehyde

[6-(5-[1,3]Dioxolan-2-yl-furan-2-yl)-quinazolin-4-yl]-(4-morpholin-4-yl-phenyl)-amine (100 mg) was dissolved in THF (10 ml) with heating, 2 MHCl (2 ml) added followed by water (10 ml) and heated at 75° until TLC (SiO₂, CH₂Cl₂/iPrOH 20%) showed deprotection was complete. The cooled reaction mixture was basified to pH 8 with 2M NaOH and the title compound isolated as a yellow solid by filtration (45 mg)

δ (DMSO) 10.07 (1H, br s); 9.72 (1H, s); 9.04 (1H, s); 8.56 (1H, s), 8.32 (1H, d, J 8.21 Hz); 7.87 (1H, d, J 8.84 Hz); 7.79 (1H, d, J 3.79 Hz); 7.67 (2H, d 8.84 Hz), 7.46 (1H, d J 3.16 Hz); 7.05 (2H, d J 8.84 Hz); 3.81 (4H, m); 3.17 (4H, m)

LC-MS ES+=401 rt 4.16

### Example 8

### {5-[4-(4-morpholin-4-yl-phenylamino)-quinazolin-6-yl]-furan-2-yl}-methanol

Reduction of 5-[4-(4-Morpholin-4-yl-phenylamino)-quinazolin-6-yl]-furan-2-carbaldehyde (450 mg) in CH₂Cl₂ (15 ml) and acetic acid (1 ml) with sodium triacetoxyborohydride (0.477 g, 2 eq) for 2 h followed by aqueous workup gave the title compound.

δ (DMSO) 9.86 (1H, br); 8.77 (1H, d, J 1.26 Hz); 8.47 (1H, s); 8.13 (1H, dd J 8.85, 1.89 Hz); 7.76 (1H, d, 8.85 Hz); 7.65 (2H, d, J 8.85 Hz); 7.05 (1H, d J 3.16 Hz); 7.0 (2H, d J 8.85 Hz); 6.50 (1H, d, 3.16 Hz), 5.76 (1H, s); 5.34 (1H, br); 4.53 (2H, s); 3.70 (4H, m); 3.10 (4H, m)

LC-MS ES+=403 rt 4.66

### Example 9

### (6-{5-[(2-Methanesulfonyl-ethylamino)-methyl]-furan-2-yl}-quinazolin-4-yl)-(4-morpholin-4-yl-phenyl)-amine

The carboxaldehyde of Example 7 (150 mg) was treated with 2-methanesulfonylethylamine (46 mg, prepared by the route in Bioorganic & Medicinal Chemistry Letters 14, 1, p 111-114 Yue-Mei Zhang et al) and 5 A molecular sieves (300 mg) at 40° in CH₂Cl₂ (15 ml) for 5 hr. Acetic acid (2 ml) and sodium triacetoxyborohydride (139 mg, 2 eq) added and stirred overnight at room temperature. The mixture was concentrated to dryness and purified by suction chromatography to give the title compound (70 mg)

δ (CDCl₃) 8.60 (1H, br); 8.29 (1H, d, 1.42 Hz); 7.88 91H, dd, J 8.68, 1.74 Hz); 7.78 (1H, d, 8.86 Hz); 7.60 (2H, d, 9.0 Hz); 6.91 (2H, d, 9.10 Hz); 6.62 (1H, d, 3.32 Hz); 6.25 (1H, d, 3.30 Hz); 3.86 (2H, s); 3.81 (4H, m); 3.22 (4H, s), 3.10 (4H, m); 2.83 (3H, s)

LC-MS ES+=508 rt 4.95

### Example 10

### {6-[5-(1,1-Dioxo-1-λ-6-thiomorpholin-4-ylmethyl)-furan-2-yl]-quinazolin-4-yl}-(4-morpholin-4-yl-phenyl)-amine

By the method of Example 9, the title compound was obtained as a yellow solid (25 mg)

δ (DMSO) 9.82 (1H, s); 8.76 (1H, s); 8.48 (1H, s); 8.14 (1H, dd, J 8.85, 1.89 Hz); 7.79 (1H, d, J 8.85 Hz); 7.64 (2H, d, J 8.85 Hz); 7.08 (1H, d, J 3.16 Hz); 7.01 (2H, d, J 8.85 Hz); 6.59 (1H, d, J 3.16 Hz); 3-85 (2H, s); 3.8 (4H, m); 3.15 (8H, m); 3.0 (4H, m) LC-MS ES+=520 it 4.85

### Example 11

### {6-[5-(4-Methyl-piperazin-1-ylmethyl)-furan-2-yl]-quinazolin-4-yl}-(4-morpholin-4-yl-phenyl)-amine

By the method of Example 9, the title compound was obtained as a yellow solid (30 mg)

δ (DMS) 9.75 (1H, s); 8.64 (1H, d, J 1.26 Hz); 8.37 (1H, s); 8.03 (1H, dd, J 8.85, 1.26 Hz); 7.86 (1H, s); 7.67 (1H, d, J 8.21 Hz); 7.54 (2H, d, 8.85 Hz); 6.96 (1H, d, J 3.16 Hz); 6.91 (2H, d, J 9.48 Hz); 6.40 (1H, d, J 3.16 Hz); 3.67 (4H, m); 3.50 (2H, s); 3.27 (8H, b); 3.02 (4H, m); 2.03 (3H, s)

LC-MS m/z 485 rt 4.48

### Example 12

### [6-(5-Morpholin-4-ylmethyl-furan-2-yl)-quinazolin-4-yl]-(4-morpholin-4-yl-phenyl)-amine

By the method of Example 9, the title compound was obtained as a pale yellow solid (20 mg)

δ (DMSO) 9.92 (1H, s); 8.80 (1H, s); 8.49 (1H, s); 8.15 (1H, d, J 8.21 Hz); 7.79 (1H, d, J 8.85 Hz); 7.65 (2H, d, J 8.85 Hz); 7.09 (1H, d, J 3.16 Hz); 7.01 (2H, d, J 8.85 Hz); 6.57 (1H, d, J 3.16 Hz); 6.53 (1H, s); 3.80 (10H, m); 3.33 (4H, m); 3.20 (4H, m)

LC-MS m/z 472 rt 4.12

### Example 13

### [6-(5-Dimethylaminomethyl-furan-2-yl)-quinazolin-4-yl]-(4-morpholin-4-yl-phenyl)-amine

By the method of Example 9, the title compound was obtained as a pale yellow solid (20 mg)

δ (DMSO) 9.93 (1H, s); 8.85 (1H, s); 8.56 (1H, s); 8.22 (1H, dd, J 8.85, 1.26 Hz); 7.86 (1H, d, J 8.85 Hz); 7.73 (2H, d, J 9.48 Hz); 7.16 (1H, d, J 3.16 Hz); 7.09 (2H, d, J 8.85 Hz); 6.63 (1H, d, J 3.16 Hz); 3.86 (4H, m); 3.73 (2H, s); 3.21 (4H, m); 2.37 (6H, s) LC-MS 430 rt 4.51

### Example 14

### [6-(5-Methylaminomethyl-furan-2-yl)-quinazolin-4-yl]-(4-morpholin4-yl-phenyl)-amine

By the method of Example 9, the title compound was obtained as a pale yellow solid (8 mg)

δ (DMSO) 9.69 (1H, s); 8.63 (1H, s); 8.35 (1H, s); 8.02 (1H, d, J 8.85 Hz); 7.64 (1H, d, J 8.85 Hz); 7.53 (2H, d, J 8.21 Hz); 6.90 (3H, m); 6.35 (1H, m); 3.65 (6H, m); 3.10 (4H, m); 2.23 (3H, s)

LC-MS m/z 4.16

### Example 15

### (4-Morpholin-4-yl-phenyl)-(6-thiophen-2-yl-quinazolin-4-yl)-amine

A suspension of (6-Iodo-quinazoline-4-yl)-(4-morpholin-4-yl-phenyl)-amine (Example 2, 2.66 g) and thiophene-2-boronic acid (Lancaster, 1.18 g, 1.5 eq) in THF (26 ml) was treated with triethylamine (2.6 ml, 3 eq.) and warmed to 60° before tris(dibenzylideneacetone)dipalladium(0) (282 mg) added and heating continued for 18 h. The resulting precipitate was isolated from the cooled reaction mixture by filtration and washed with THF (10 ml). The solid was dissolved in DMSO (40 ml) and passed through a pad of Celite before being diluted with water and the precipitate isolated by filtration and dried in vacuo to give the title compound as a yellow solid (1.2 g, 50%)

δ (DMSO) 10.75 (1H, br s); 8.9 (1H, s); 8.61 (1H, s); 8.20 (1H, dd, J 8.85, 1.89 Hz); 7.8 (2H, d, 8.85 Hz); 7.76 (1H, s); 7.66 (1H, d, J 4.42 Hz); 7.58 (2H, d, J 8.85 Hz), 7.21 (1H, t, 3.79 Hz); 7.0 (2H, d, J 8.85 Hz); 3.70 (4H, m); 3.10 (4H, m)

LC-MS ES+=389 rt 4.32

### Example 16

### (6-Chloro-quinazolin-4-yl)-(4-morpholin-4-yl-phenyl)-amine

By a similar method to example 15 was obtained the title compound (42 mg)

δ (DMSO) 11.86 (1H, s); 9.26 (1H, s); 8.97 (1H, s); 8.5 (1H, dd, J 8.85, 1.9 Hz); 8.06 (2H, d 8.85 Hz); 7.73 (2H, d, J 8.85 Hz); 7.67 (2H, d, J 8.85 Hz); 7.28 (1H, d, J 8.85 Hz); 7.16 (1H, d, J 9.48 Hz); 7.11 (1H, d, J 9.48 Hz); 3.81 (4H, m); 3.2 (4H, m)

LC-MS ES+=417.5 rt 4.4

### Example 17

### (4-Morpholin-4-yl-phenyl)-(6-o-tolyl-quinazolin-4-yl)-amine

By a similar method to example 15 was obtained the title compound (25 mg)

δ (MeOD) 8.58 (1H, s); 8.44 (1H, d, J 1.26 Hz); 7.93 (1H, d, J 1.89 Hz); 7.91 (1H, s); 7.65 (2H, d, J 8.85 Hz); 7.39 (4H, m); 7.10 (2H, d, J 8.85 Hz); 6.96 (2H, s); 3.92 (2H, m); 3.24 (4H, m); 2.39 (3H, s)

LC-MS ES+=397 rt 4.26

### Example 18

### (4-Morpholin-4-yl-phenyl)-(6-thiazol-2-yl-quinazolin-4-yl)-amine

A similar method to example 5 gave the title compound (5.3 mg)

δ (DMSO) 10.16 (1H, s), 9.17 (1H, s), 8.61 (1H, s), 8.5 (1H, d), 8.48 (1H, d, J 8.8 Hz), 8.10 (1H, d, J 3 Hz), 7.98 (1H, d, J 3 Hz), 7.91 (1H, d, 8.8 Hz), 7.72 (2H, d, J 8.8 Hz), 7.09 (2H, d, J8.8 Hz), 3.85 (4H, m), 3.2 (4H, m)

LC-MS m/z 390 rt 3.31

### Example 19

### [6-(2-Methoxy-pyrimidin-5-yl)-quinazolin-4-yl]-(4-morpholin-4-yl-phenyl)-amine

By a similar method to Example 15, the title compound was obtained as a yellow solid (120 mg)

δ (DMSO) 9.75 (1H, s), 9.15 (2H, s), 8.86 (1H, s), 8.53 (1H, s), 8.23 (1H, d, J 8.85 Hz), 7.83 (1H, d, J 8.85 Hz), 7.64 (2H, d, J 8.85 Hz), 6.996 (2H, d, J 8.85 Hz), 4.0 (3H, s), 3.76 (4H, m), 3.11 (4H, m)

LC-MS m/z 415

### Example 20

### [6-(4-Methyl-thiophen-2-yl)-quinazolin-4-yl]-(4-morpholin-4-yl-phenyl)-amine

By a similar method to Example 15, the title compound was obtained as a red solid.

δ (DMSO) 11.8 (1H, br s), 9.18 (1H, s), 8.83 (1H, s), 8.31 (1H, d, J 8.85 Hz), 7.97 (1H, d, J 8.2 Hz), 7.77 (1H, s), 7.61 (2H, d, J 8.85 Hz), 7.32 (1H, s), 7.08 (1H, s), 3.78 (4H, m), 3.19 (4H, m), 2.3 (3H, s)

LC-MS m/z 403

### Example 21

### 5-[4-(4-Morpholin-4-yl-phenylamino)-quinazolin-6-yl]-thiophene-2-carboxylic acid

By a similar method to Example 15, the title compound was obtained as a red-brown solid.

δ (DMSO) 11.97 (1H, br s), 9.36 (1H, s), 8.86 (1H, s), 8.43 (1H, d, J 8.2 Hz), 7.99 (2H, m), 7.82 (1H, d, J 3.8 Hz), 7.61 (2H, d, J 8.85 Hz), 7.08 (2H, d, J 9.5 Hz), 3.77 (4H, m), 3.18 (4H, m)

LC-MS m/z 433

### Example 22

### [6-(4-Methanesulfonyl-phenyl)-quinazolin-4-yl]-(4-morpholin-4-yl-phenyl)-amine

By a similar method to Example 15, the title compound was obtained as a yellow solid

δ (DMSO) 9.91 (1H, br s), 8.91 (1H, s), 8.53 (1H, s), 8.15 (4H, m), 7.86 (1H, d, J 8.85 Hz), 7.64 (2H, d, J 8.85 Hz), 6.99 (2H, d, J 8.85 Hz), 3.76 (4H, m), 3.29 (3H, s), 3.11 (4H, m)

LC-MS M/z 461

### Example 23

### (4-Morpholin-4-yl-phenyl)-[6-(3-pyrazol-1-yl-phenyl)-quinazolin-4-yl]-amine

By a similar method to Example 15, the title compound was isolated as a brown solid by preparative LC-MS (5 mg), δ (DMSO) 3.12 (s, 4H) 3.75 (s, 4H) 6.25 (s, 1H) 7.01 (d, 2H) 7.54 (m, 4H) 7.65 (d, 2H) 7.89 (d, 1H) 8.11 (m, 2H) 8.55 (s, 1H) 8.72 (s, 1H) 9.99 (s, 1H), LC/MS RT=3.62 min Found ES+=449.4

### Example 24

### 4-[4-(4-Morpholin-4-yl-phenylamino)-quinazolin-6-yl]-benzonitrile

By a similar method to Example 15, the title compound was isolated as a brown solid by preparative LC-MS (5 mg). δ (DMSO) 3.12 (s, 4H) 3.77 (s, 4H) 7.01 (d, 2H) 7.62 (d, 2H) 7.82 (d, 2H) 8.03-8.24 (m, 5H) 8.50 (s, 1H) 8.90 (s, 1H) 9.91 (bs, 1H), LC/MS RT=2.50 min Found ES+=408.5

### Example 25

### Furan-2-carboxylic acid [4-(4-morpholin-4-yl-phenylamino)-quinazolin-6-yl]-amide

Step 1: (4-Morpholin-4-yl-phenyl)-(6-nitro-quinazolin-4-yl)-amine (prepared by the method of Example 1, 3.60 LC-MS ES+352 ES−350, 0.5 g) was added to a stirred solution of THF:MeOH (1:1, 25 ml) followed by Raney nickel (2 spatula, excess). The mixture was heated to 50° C. at which stage hydrazine (1 ml) was added and left to stir for 5 hr at this temperature. The mixture was allowed to cool and filtered through celite. The solvent was removed under vacuum to afford a solid which was purified by column chromatography on silica using 2.5-5% MeOH:DCM. Yield 0.16 g (35%).

LCMS: RT 2.01, ES⁺322, ES⁻320

δ (DMSO) 9.28 (1H, s); 8.25 (1H, s); 7.67 (2H, d, J8.85 Hz); 7.50 (1H, d, J8.85 Hz); 7.34 (1H, d, 1.90 Hz), 7.22 (1H, dd, J8.85 Hz, 1.90 Hz); 6.96 (2H, d, J8.85 Hz); 5.53 (2H, s); 3.77 (4H, m), 3.09 (4H, m).

Step 2: To a portion of the amine (50 mg) in pyridine (4 ml) was added 2-furoyl chloride (0.033 g, 1.2 eq) and stirred for 4 h. The pyridine was removed in vacuo and the residue purified by chromatography to give the title compound

δ (DMSO) 8.77 (1H, s); 8.43 (1H, s); 8.01 (1H, d, J8.5 Hz); 7.97 (1H, s); 7.72 (1H, d, J8.85 Hz); 7.64 (2H, d, J8.85 Hz); 7.42 (1H, d, J3.16 Hz); 6.96 (2H, d, J8.85 Hz); 6.73 (1H, m); 3.75 (4H, m); 3.08 (4H, m), LC-MS m/z 416 rt 2.22

### Example 26

### 4-(4-Morpholin-4-yl-phenylamino)-quinazoline-6-carboxylic acid 4-methoxy-benzylamide

Step 1: Preparation of 4-(4-morpholin-4-yl-phenylamino)-quinazoline-6-carboxylic acid

A solution of Example 2 (2.0 g, 4.6 mMol), potassium carbonate (1.4 g, 11.6 mMol), tetrakis(triphenylphosphine)-palladium(0) (0.27 g, 0.23 mMol) and dichlorobis-(triphenylphosphine)palladium(II) (0.16 g, 0.23 mMol) in 4:1 dimethylformamide:water (75 ml) was placed under an atmosphere of carbon monoxide and heated to 100° C. for 6 hours. On cooling, the reaction solvent was removed under vacuum and the residue taken into water. On acidification to pH2, with 1M hydrochloric acid, an orange precipitate formed. This was filtered off and slurried in methanol. The resulting suspension was filtered and air-dried to give 4-(4-morpholin-4-yl-phenylamino)-quinazoline-6-carboxylic acid as a yellow solid. Yield=0.56 g (1.6 mMol, 35%)

LC/MS: RT−2.05, MH⁺ 351, δ (DMSO) 10.37 (1H, s); 9.46 (1H, s); 8.79 (1H, s); 8.49 (1H, dd, J 8.4 Hz, 1.2 Hz); 8.01 (1H, d, J 8.2 Hz); 7.87 (2H, d, J 8.8 Hz); 7.21 (2H, d, J 8.8 Hz); 3.98 (4H, t, J 4.4 Hz); 3.33 (4H, t, J 4.4 Hz)

Step 2: A solution of 4-(4-morpholin-4-yl-phenylamino)-quinazoline-6-carboxylic acid (50 mg, 0.14 mMol), O-(7-azabenzotriazol-1-yl)-N—N—N′—N′-tetramethyluronium hexafluorophosphate (55 mg, 0.14 mMol) and triethylamine (45 μl, 0.32 mMol) in dimethylformamide (2 ml) was treated with 4-methoxybenzylamine and stirred overnight. On addition of water to the reaction mixture, a precipitate developed. This was collected by filtration and air-dried to give the title compound

δ (DMSO) 10.06 (1H, s); 9.20 (1H, s); 9.16 (1H, s); 8.36 (1H, d, J 8.2 Hz); 7.92 (1H, d, J 8.8 Hz); 7.78 (2H, d, J 8.8 Hz); 7.45 (2H, d, J 8.8 Hz); 7.12 (2H, d, J 8.8 Hz); 7.05 (2H, d, J 8.2 Hz); 4.62 (2H, d, J 5.1 Hz); 3.85-3.94 (7H, m); 3.24 (4H, t, J 3.6 Hz), LC-MS m/z 470 rt 2.46

Prepared in a similar fashion to Example 26, using the appropriate amine (1.2 eq) either pure or as a solution in tetrahydrofuran, (where available) in Step 2 were:

### Example 27

### 3-{[4-(4-Morpholin-4-yl-phenylamino)-quinazoline-6-carbonyl]-amino}-benzoic acid ethyl ester

δ (DMSO) 10.68 (1H, s); 10.00 (1H, s); 9.13 (1H, s); 8.57 (1H, s); 8.47 (1H, s); 8.30 (1H, dd, J 8.8 Hz, 1.3 Hz); 8.13 (1H, d, J 8.2 Hz); 7.84 (1H, d, J 8.8 Hz); 7.72 (1H, d, J 8.2 Hz); 7.67 (2H, d, J 8.8 Hz); 7.50-7.59 (1H, m); 6.99 (2H, d, J 8.8 Hz); 4.33 (2H, q, J 7.2 Hz); 3.75 (4H, t, J 4.4 Hz); 3.10 (4H, t, J 4.4 Hz), 1.33 (3H, t, J 6.9 Hz), LC-MS m/z 498 rt 2.70

### Example 28

### 4-(4-Morpholin-4-yl-phenylamino)-quinazoline-6-carboxylic acid 3-methoxy-benzylamide

δ (DMSO) 9.75 (1H, s); 8.93 (1H, t, J 5.7 Hz); 8.85 (1H, s); 8.35 (1H, s); 8.05 (1H, d, J 8.2 Hz); 7.76 (1H, s); 7.60 (1H, d, J 8.2 Hz); 7.46 (2H, d, J 8.2 Hz); 7.07 (1H, t, J 7.6 Hz); 6.72-6.82 (3H, m); 6.63 (1H, d, J 8.8 Hz); 4.34 (2H, d, J 5.1 Hz); 3.11-3.17 (7H, m); 2.87-2.94 (3H, m), LC-MS m/z 470 rt 2.48

### Example 29

### 4-(4-Morpholin-4-yl-phenylamino)-quinazoline-6-carboxylic acid 4-methyl-benzylamide

δ (DMSO) 10.14 (1H, s); 9.30 (1H, t, J 6.1 Hz); 9.24 (1H, s); 8.76 (1H, s); 8.44 (1H, dd, J 8.8 Hz, 1.3 Hz); 8.00 (1H, J 8.8 Hz); 7.87 (2H, d, J 8.8 Hz); 7.47 (2H, d, J 8.2 Hz); 7.36 (2H, d, J 7.6 Hz); 7.20 (2H, d, 8.8 Hz); 4.73 (2H, d, J 5.7 Hz); 3.94-4.00 (4H, m); 3.29-3.35 (4H, m); 2.49 (3H, s) LC-MS m/z 454 rt 2.58

### Example 30

### 4-(4-Morpholin-4-yl-phenylamino)-quinazoline-6-carboxylic acid methylamide

δ (DMSO) 9.81 (1H, s); 8.86 (1H, s); 8.39-8.45 (2H, m); 8.04 (1H, dd, J 8.8 Hz, 1.9 Hz); 7.65 (1H, d, J 8.2 Hz); 7.53 (2H, d, J 8.8 Hz); 6.86 (2H, d, J 8.8 Hz); 3.59-3.67 (4H, m); 2.95-3.01 (4H, m); 2.73 (3H, d, J 4.4 Hz), LC-MS rt 2.08, m/z 364

### Example 31

### 4-(4-Morpholin-4-yl-phenylamino)-quinazoline-6-carboxylic acid dimethylamide

δ (DMSO) 9.60 (1H, s); 8.43 (1H, s); 8.35 (1H, s); 7.63 (1H, dd, J 8.2 Hz, 1.3 Hz); 7.57 (2H, d, J 8.8 Hz); 6.79 (2H, d, J 8.8 Hz); 3.53-3.59 (4H, m); 2.89-2.94 (4H, m); 2.86 (3H, s); 2.79 (3H, s), LC-MS rt 2.09, m/z 378

### Example 32

### 4-(4-Morpholin-4-yl-phenylamino)-quinazoline-6-carboxylic acid ethylamide

δ (DMSO) 9.96 (1H, s); 9.01 (1H, s); 8.63 (1H, t, J 5.4 Hz); 8.57 (1H, s); 8.21 (1H, dd, J 8.8 Hz, 1.9 Hz); 7.80 (1H, d, J 8.8 Hz); 7.69 (2H, d, J 9.5 Hz); 7.02 (2H, d, J 8.8 Hz); 3.75-3.82 (4H, m); 3.38 (2H, q, J 6.9 Hz); 3.11-3.17 (4H, m); 1.21 (3H, t, J 6.9 Hz), LC-MS rt 2.15, m/z 378

### Example 33

### N-{3-[4-(4-Morpholin-4-yl-phenylamino)-quinazolin-6-yl]-phenyl}-acetamide

By a similar method to Example 15, the title compound was isolated as a brown solid by preparative LC-MS (5 mg).

δ (DMSO) 2.10 (s, 3H) 3.13 (s, 4H) 3.77 (s, 4H) 7.01 (d, 2H) 7.54 (m, 2H) 7.67 (d, 2H) 7.85 (d, 1H) 8.03 (m, 2H) 8.53 (s, 1H) 8.79 (s, 1H) 9.89 (s, 1H) 10.15 (s, 1H)

LC/MS RT=3.62 min Found ES+=449.4

### Example 34

### {4-[4-(4-Morpholin-4-yl-phenylamino)-quinazolin-6-yl]-phenyl}-carbamic acid benzyl ester

By the method of Example 15, δ (DMSO) 3.10-3.14 (t, 2H), 3.75-3.93 (t, 2H), 5.19 (s, 2H), 6.99-7.03 (d, 2H, J=9 Hz), 7.36-7.48 (m, 5H), 7.64-7.68 (t, 3H), 7.78-7.87 (m, 3H), 8.13-8.16 (dd, 1H), 8.33-8.35 (d, 1H), 8.50 (s, 1H), 8.77 (s, 1H), 9.82 (s, 1H), 9.97 (s, 1H); LC-MS m/z 533, rt 2.81

### Example 35

### N-{4-[4-(4-Morpholin 4-yl-phenylamino)-quinazolin-6-yl]-phenyl}-acetamide

By the method of Example 15, δ (DMSO) 2.17-2.19 (s, 3H), 3.21-3.24 (t, 4H), 3.85-3.89 (t, 4H), 7.10-7.13 (d, 2H), 7.75-7.79 (d, 2H), 7.84-7.98 (m, 5H), 8.24-8.27 (d, 1H), 8.60 (s, 1H), 8.88 (s, 1H), 9.92 (s, 1H), 10.23 (s, 1H), LC-MS m/z 440, rt 2.44

### Example 36

### {4-[4-(4-Morpholin-4-yl-phenylamino)-quinazolin-6-yl]-phenyl}-carbamic acid tert-butyl ester

By the method of Example 15, δ 1.47 (s, 9H), 3.06-3.10 (t, 4H), 3.71-3.74 (t, 4H), 6.95-6.99 (d, 2H), 7.57-7.64 (m, 4H), 7.76-7.79 (t, 3H), 8.10-8.12 (d, 1H), 8.45 (s, 1H), 8.75 (s, 1H), 9.50 (s, 1H), 9.75 (s, 1H); LC-MS m/z 498, rt 2.86

### Example 37

### {3-[4-(4-Morpholin-4-yl-phenylamino)-quinazolin-6-yl]-benzyl}-carbamic acid tert-butyl ester

By the method of Example 15, δ 1.17 (s, 3H), 2.87-2.90 (t, 4H), 3.51-3.55 (t, 4H), 4.01-4.03 (d, 2H), 6.75-6.79 (d, 2H), 7.27-7.31 (d, 2H), 7.41-7.44 (t, 2H), 7.49-7.51 (d, 1H), 7.58-7.62 (d, 1H), 7.87 (d, 1H), 7.9 (d, 1H), 8.2 (s, 1H), 8.28 (s, 1H), 8.56 (s, 1H), 9.65 (s, 1H), LC-MS m/z 514, rt 2.18

### Example 38

### N-{3-[4-(4-Morpholin-4-yl-phenylamino)-quinazolin-6-yl]-phenyl}-methanesulfonamide

By the method of Example 15

δ (DMSO,) 2.86-2.89 (s, 3H), 2.95-2.98 (t, 4H), 3.53-3.57 (t, 4H), 6.86-6.89 (d, 2H), 7.07-7.10 (d, 1H), 7.31-7.45 (m, 5H), 7.88 (d, 1H), 8.07-8.10 (d, 1H), 8.65 (s, 1H), 9.05 (s, 1H), 9.35 (s(broad), 1H), 9.78 (s, 1H), 11.79 (s, 1H), LC-MS m/z 476 rt 2.54

### Example 39

### (6-Iodo-quinazolin-4-yl)-(4-morpholin-4-yl-2-trifluoromethyl-phenyl)-amine

Step 1: A solution of 5-fluoro-2-nitrobenzotrifluoride (2.1 g, 10 mM) and triethylamine (2.50 ml, 24 mMol) in acetonitrile (35 ml) was treated with morpholine (1.74 ml, 20 mMol). The resulting solution was heated at reflux overnight. On cooling, the reaction solvent was removed under vacuum and the crude residue partitioned between dichloromethane and 10% (w/v) citric acid solution. The organics were separated, dried over magnesium sulphate and reduced under vacuum to give 4-(4-nitro-3-trifluoromethyl-phenyl)-morpholine (2.63 g, 95%), LC/MS: RT−3.69, no ionization δ (CDCl₃) 8.04 (1H, d, J 8.8 Hz); 7.17 (1H, d, J 3.2 Hz); 6.96 (1H, dd, J 8.8 Hz, 2.5 Hz); 3.89 (4H, t, J 4.5 Hz); 3.40 (4H, t, J 4.5 Hz)

Step 2: A suspension of 4-(4-nitro-3-trifluoromethyl-phenyl)-morpholine (2.63 g, 0.95 mMol) and 10% palladium on carbon (263 mg) in 2:1 toluene:ethanol (75 ml) was placed under an atmosphere of hydrogen, using standard procedures, until reaction was complete. The reaction mixture was filtered through a pad of celite, which was then washed with ethanol. The filtrates were reduced under vacuum to give 4-morpholin-4-yl-2-trifluoromethyl-phenylamine as a beige solid. (1.25 g, 53%), LC/MS: RT−2.40, no ionization, δ (CDCl₃) 7.05-7.12 (2H, m); 6.83 (1H, d, J 8.8 Hz); 3.96 (4H, t, J 4.4 Hz); 3.14 (4H, t, 4.4 Hz)

Step 3: Treatment of the product from step 2 (280 mg) with 4-chloro-6-iodo-quinazoline (300 mg, 1.1 mMol) in acetonitrile (4 ml) at reflux overnight, gave a precipitate. The reaction was cooled and the precipitate filtered off. This was washed with 1M sodium hydroxide solution and water before being air dried to the title compound δ (DMSO) 9.79 (1H, s); 8.85 (1H, s); 8.27 (1H, d, J 12.0 Hz); 8.03 (1H, d, J 7.0 Hz); 7.17-7.43 (4H, m); 3.72-3.78 (4H, m); 3.16-3.23 (4H, m), LC/MS: RT−2.80, MH⁺ @ 501,

### Example 40

### (4-Morpholin-4-yl-2-trifluoromethyl-phenyl)-(6-thiophen-2-yl-quinazolin-4-yl)-amine

A solution of Example 39 (170 mg, 0.4 mMol), 2-thiopheneboronic acid (50 mg, 0.4 mMol), triethylamine (120 μl, 1.0 mMol) and tris(dibenzylideneacetone)-dipalladium(0) (50 mg, 15 Mol %) in anhydrous tetrahydrofuran (3 ml) was heated at reflux overnight. On cooling, the reaction mixture was reduced onto silica and flash chromatography (eluting with a dichloromethane −2.5% methanol in dichloromethane gradient) gave the title compound as a yellow solid.

δ (DMSO) 9.84 (1H, s); 8.71 (1H, s); 8.31 (1H, s); 8.09 (1H, d, J 8.2 Hz); 7.73 (1H, d, J 8.8 Hz); 7.65 (1H, d, J 3.2 Hz); 7.60 (1H, d, J 5.1 Hz); 7.14-7.36 (4H, m); 3.67-3.77 (4H, m); 3.13-3.19 (4H, m). LC/MS: RT-2.80, MH⁺ @ 457

### Example 41

### (6-Iodo-quinazolin-4-yl)-(3-methoxy-4-morpholin-4-yl-phenyl)-amine

Step 1: 2-bromo-5-nitro-anisole (0.5 g) and morpholine (1.92 g) were heated together at 130° overnight. The cooled reaction mixture was added to ice and the resulting precipitate washed with water and dried to give the desired 4-(2-methoxy-4-nitro-phenyl)-morpholine (91%, m/z 239)

Step 2: Hydrogenation of the nitro group using palladium on carbon as catalyst at rt in ethanol gave 3-methoxy-4-morpholin-4-yl-phenylamine as a brown solid (76%, 0.159 g) which was used without further purification.

Step 3: Heating of 3-methoxy-4-morpholin-4-yl-phenylamine (136 mg) and 4-chloro-6-iodoquinazoline (186 mg) in acetonitrile (10 ml) at reflux overnight gave, on cooling, a precipitate that was isolated by filtration, washed with water then slurried with 1N NaOH and washed with further water and dried. This gave the title compound (263 mg, 88%)

δ (DMSO) 11.01 (1H, br s); 9.20 (1H, s); 8.83 (1H, s); 8.29 (1H, d, J8.85 Hz); 7.67 (1H, d, J8.85 Hz); 7.40 (2H, m); 6.98 (1H, d, J8.85 Hz); 8.83 (3H, s); 3.76 (4H, m); 3.01 (4H, m), LC-MS rt 2.74, m/z E+463

### Example 42

### (3-Methoxy-4-morpholin-4-yl-phenyl)-(6-thiophen-2-yl-quinazolin-4-yl)-amine

A similar method to Example 15, using Example 41 as starting material gave the title compound (14 mg, 11%)

δ (DMSO) 9.96 (1H, s); 8.86 (1H, s); 8.61 (1H, s); 8.22 (1H, d, J8.85 Hz); 7.87 (1H, d, J8.85 Hz); 7.83 (1H, d, J3.79 Hz); 7.76 (1H, d, J5.06 Hz); 7.48 (2H, m); 7.33 (1H, t J5.05, 3.79 Hz); 7.03 (1H, J8.85 Hz); 3.92 (3H, s); 3.84 (4H, m); 3.07 (4H, m), LC-MS rt 3.63, m/z E+419

### Example 43

### (2-Methyl-4-morpholin-4-yl-phenyl)-(6-thiophen-2-yl-quinazolin-4-yl)-amine

Step 1: A solution of 5-fluoro-2-nitrotoluene (1.22 ml, 10 mM) and triethylamine (2.10 ml, 20 mMol) in acetonitrile (30 ml) was treated with morpholine (1.74 ml, 20 mMol). The resulting solution was heated at reflux overnight. On cooling, the reaction solvent was removed under vacuum and the crude residue partitioned between dichloromethane and 10% (w/v) citric acid solution. The organics were separated, dried over magnesium sulphate and reduced under vacuum to give 4-(3-methyl-4-nitrophenyl)-morpholine. (1.96 g, 88%) LC/MS: RT−2.76, no ionization

Step 2: A suspension of 4-(3-methyl-4-nitrophenyl)-morpholine 1.96 g, (8.9 mMol) and 10% palladium on carbon (100 mg) in toluene (30 ml) was placed under an atmosphere of hydrogen, using standard procedures, until reaction was complete. The reaction mixture was filtered through celite and the liquors reduced under vacuum to give 2-methyl-4-morpholin-4-yl-phenylamine as a dark brown solid. (1.07 g, 63%), LC/MS: RT−0.71, MH⁺ @ 193; δ (CDCl₃) 6.74 (1H, s); 6.64-6.70 (2H, m); 3.88 (4H, t, J 4.5 Hz); 3.06 (4H, t, J 4.5 Hz); 1.99 (3H, s)

Step 3: A suspension of 4-chloro-6-iodo-quinazoline (500 mg, 1.8 mMol) and 2-methyl-4-morpholin-4-yl-phenylamine (360 mg, 1.9 mMol) in acetonitrile (3 ml) was heated at reflux overnight, during which a precipitate developed. The reaction was cooled and the precipitate filtered off. This was washed with 1M sodium hydroxide solution and water before being air dried to give (6-iodo-quinazolin-4-yl)-(2-methyl-4-morpholin-4-yl-phenyl)-amine (738 mg, 95%). LC/MS: RT−3.55, MH⁺ @ 447

δ (DMSO) 10.35 (1H, br s); 9.04 (1H, d, 1.3 Hz); 8.55 (1H, s); 8.20 (1H, dd, J 8.8 Hz, 1.3 Hz); 7.60 (1H, d, J 8.8 Hz); 7.14 (1H, d, J 8.8 Hz); 6.92 (1H, d, J 2.5); 6.87 (1H, dd, J 8.2 Hz, 1.9 Hz); 3.77 (4H, t, 4.4 Hz); 3.15 (4H, t, 4.4 Hz); 2.15 (3H, s)

Step 4: A solution of (6-iodo-quinazolin-4-yl)-(2-methyl-4-morpholin-4-yl-phenyl)-amine (250 mg, 0.6 mMol), 2-thiopheneboronic acid (75 mg, 0.6 mMol), triethylamine (150 μl, 1.2 mMol) and tris(dibenzylideneacetone)dipalladium(0) (50 mg, 10 Mol %) in anhydrous tetrahydrofuran (10 ml) was heated at reflux overnight. On cooling, the reaction mixture was reduced onto silica and flash chromatography (eluting with a 200:8:1 dichloromethane:ethanol:aqueous ammonia mixture) gave (2-methyl-4-morpholin-4-yl-phenyl)-(6-thiophen-2-yl-quinazolin-4-yl)-amine as a yellow solid. LC/MS: RT−3.54, MH⁺ @ 403; δ (DMSO) 9.89 (1H, s); 8.89 (1H, d, J 1.9 Hz); 8.46 (1H, s); 8.25 (1H, dd, J 8.8 Hz, 1.9 Hz); 7.89 (1H, d, J 8.8 Hz); 7.84 (1H, 3.8 Hz), 7.78 (1H, d, J 5.0 Hz); 7.36 (1H, dd, J 5.0 Hz, 3.8 Hz); 7.30 (1H, d, 8.2 Hz); 7.05 (1H, d, 1.9 Hz); 6.98 (1H, dd, J 8.8 Hz, 3.2 Hz); 3.90 (4H, t, J 4.4 Hz); 3.28 (4H, t, J 4.4 Hz); 2.29 (3H, s)

### Example 44

### (3-Methyl-4-morpholin-4-yl-phenyl)-(6-thiophen-2-yl-quinazolin-4-yl)-amine

Step 1: A solution of 2-fluoro-5-nitrotoluene (1.65 g, 10.6 mMol), triethylamine (2.96 ml, 21.2 mMol) and morpholine (1.86 ml, 21.2 mMol) in acetonitrile (30 ml) was heated at reflux overnight. On cooling, the reaction solvent was reduced under vacuum and the residue taken into morpholine. This solution was heated at 100° C. until the reaction was complete by TLC. The reaction mixture was diluted with dichloromethane and washed with 1M citric acid solution. The organics were dried over magnesium sulphate and reduced under vacuum to give an oil. This was further purified by flash chromatography (eluting with a petrol—9:1 petrol:ethyl acetate gradient) to give 4-(2-methyl-4-nitro-phenyl)-morpholine as an orange solid. (0.53 g, 22%), LC/MS: RT−3.76, no ionization; δ (CDCl₃) 7.99-8.02 (2H, m); 6.94 (1H, d, J 9.5 Hz); 3.81 (4H, t, J 4.5 Hz); 2.95 (4H, t, J 4.5 Hz); 2.31 (3H, s)

Step 2: A suspension of 4-(2-methyl-4-nitro-phenyl)-morpholine (0.53 g, 2.4 mMol) and 10% palladium on carbon (55 mg) in 1:1 toluene:ethanol (25 ml) was placed under an atmosphere of hydrogen, using standard procedures, until reaction was complete. The reaction mixture was filtered through a pad of celite, which was then washed with ethanol. The filtrates were reduced under vacuum to give 3-methyl-4-morpholin-4-yl-phenylamine as a tan solid. (0.47 g, 100%), LC/MS: RT−2.66, MH⁺ @ 193; δ (CDCl₃) 6.82 (1H, d, J 8.2 Hz); 6.48-6.54 (2H, m); 3.76 (4H, t, J 4.4 Hz); 2.76 (4H, t, J 4.4 Hz); 2.18 (3H, s)

Step 3: A suspension of 4-chloro-6-iodo-quinazoline (300 mg) and 3-methyl-4-morpholin-4-yl-phenylamine (240 mg) in acetonitrile (4 ml) was heated at reflux overnight, during which a precipitate developed. The reaction was cooled and the precipitate filtered off. This was washed with 1M sodium hydroxide solution and water before being air dried to give (6-iodo-quinazolin-4-yl)-(3-methyl-4-morpholin-4-yl-phenyl)-amine. LC/MS: RT−2.81, MH⁺ @ 447; δ (DMSO) 9.82 (1H, s); 9.03 (1H, d, J 1.9 Hz); 8.59 (1H, s); 8.12 (1H, dd, J 8.8 Hz, 1.9 Hz); 7.60-7.70 (2H, m); 7.58 (1H, d, J 8.8 Hz); 7.09 (1H, d, J 8.8 Hz); 3.78 (4H, t, J 4.1 Hz); 2.87 (4H, t, J 4.1 Hz); 2.32 (3H, s)

Step 4: A solution of (6-iodo-quinazolin-4-yl)-(3-methyl-4-morpholin-4-yl-phenyl)-amine (170 mg, 0.4 mMol), 2-thiopheneboronic acid (50 mg, 0.4 mMol), triethylamine (120 μl, 1.0 mMol) and tris(dibenzylideneacetone)dipalladium(0) (50 mg, 15 Mol %) in anhydrous tetrahydrofuran (3 ml) was heated at reflux overnight. On cooling, the reaction mixture was reduced onto silica and flash chromatography (eluting with a dichloromethane—2.5% methanol in dichloromethane gradient) gave (3-methyl-4-morpholin-4-yl-phenyl)-(6-thiophen-2-yl-quinazolin-4-yl)-amine as a yellow solid.

LC/MS: RT−2.76, MH⁺ @ 403; δ (DMSO) 10.05 (1H, s); 8.97 (1H, d, J 1.9 Hz); 8.71 (1H, s); 8.32 (1H, dd, J 8.8 Hz, 1.9 Hz); 7.99 (1H, d, J 8.8 Hz); 7.93 (1H, dd, J 3.8 Hz, 1.3 Hz); 7.87 (1H, dd, J 5.1 Hz, 1.3 Hz); 7.75-7.85 (3H, m,); 7.44 (1H, dd, J 5.1 Hz, 3.2 Hz); 7.29 (1H, d, J 8.8 Hz); 3.96 (4H, t, J 4.4 Hz); 3.06 (4H, t, J 4.4 Hz); 2.51 (3H, s)

### Example 45

### Ethyl-(4-morpholin-4-yl-phenyl)-(6-thiophen-2-yl-quinazolin-4-yl)-amine

Step 1: To a stirred solution of dry DMF (10 ml) was added N-(4-aminophenyl)morpholine (0.5 g, 2.80 mmol) followed by Et₃N (0.70 g, 7.0 mmol). Acetyl chloride (0.24 g, 3.10 mmol) was added slowly and the mixture stirred at room temperature overnight. Water (50 ml) was added and the mixture was extracted with ethyl acetate (2×20 ml). The organic washings were combined and dried (Na₂SO₄) and the solvent was removed under vacuum to afford N-(4-morpholin-4-yl-phenyl)-4-acetamide as a solid. Yield 0.29 g (47%). δ (DMSO) 9.71 (1H, bs); 7.42 (2H, d, J8.85 Hz); 6.87 (2H, d, J9.48 Hz); 3.72 (4H, m); 3.01 (4H, m); 1.98 (3H, s), LCMS: RT 1.97, ES⁺ 221

Step 2: Treatment of N-(4-morpholin-4-yl-phenyl)-4-acetamide (1 g) in a similar manner to Example 46, Step 2, gave semi crude ethyl-(4-morpholin-4-yl-phenyl)-amine (0.92 g,) which was reacted with 4-chloro-6-iodoquinazoline as per Example 46, Step 3 and then reacted as per Example 44, Step 4 to give the title compound (104 mg, 61%) δ (DMSO) 8.61 (1H, s); 8.18 (1H, s); 7.93 (1H, d, J8.85 Hz); 7.70 (2H, d, J8.85 Hz); 7.50 (1H, d, J4.42 Hz); 7.14 (5H, m); 4.11 (2H, m); 3.76 (4H, m); 3.19 (4H, m); 1.22 (3H, t, J6.31 Hz), LC-MS rt 2.67, m/z E+417

### Example 46

### (6-Iodo-quinazolin-4-yl)-methyl-(4-morpholin-4-yl-phenyl)amine

Step 1: Formic acid (0.41 g) was added to acetic anhydride (0.75 g) with stirring at 0° then heated to 50° for 2 h. The cooled mixture was diluted with dry THF (5 ml) and 4-morpholinoaniline (0.5 g) added and the mixture returned to for 3 h. The solvent was removed in vacuo to give N-(4-morpholin-4-yl-phenyl)-4-formamide as a yellow solid (450 mg, 77%), δ (DMSO) 8.72 (1H, s), 7.90 (1H, d, J8.85 Hz); 7.51 (1H, J8.85 Hz); 7.15 (5H, m); 3.81 (4H, m); 3.56 (3H, s); 3.21 (4H, m), LC-MS rt 2.62, m/z E+447.

Step 2: A solution of N-(4-morpholin-4-yl-phenyl)-4-formamide (0.29 g) in dry THF (2 ml) was treated with sodium borohydride (160 mg) at 0° and stirred for 30 minutes. A solution of BF₃/Et₂O (0.67 ml) was added over a period of 10 min and stirred for a further 1 hr at 0° C. The mixture was then heated at reflux for 5 hr. The mixture was cooled and water (3 ml) was added dropwise to hydrolyse excess sodium borohydride. The mixture was extracted with diethyl ether (2×10 ml). The organic washings were combined dried (Na₂SO₄) and the solvent was removed under vacuum to afford methyl-(4-morpholin-4-yl-phenyl)-amine as a white solid. Yield 43 mg (16%) δ (DMSO) 6.76 (2H, d, J8.85 Hz); 6.47 (2H, d, J8.85 Hz); 5.17 (1H, bs); 3.69 (4H, m); 2.87 (4H, m); 2.60 (3H, s), LCMS: RT 2.35, ES⁺ 193

Step 3: Heating of methyl-(4-morpholin-4-yl-phenyl)-amine (37 mg) and 4-chloro-6-iodoquinazoline (52 mg) in acetonitrile (6 ml) at reflux overnight gave, on cooling, a precipitate that was isolated by filtration, washed with water then slurried with 1N NaOH and washed with further water and dried. This gave the title compound (22 mg, 28%) δ(DMSO) 8.72 (1H, s), 7.90 (1H, d, J8.85 Hz); 7.51 (1H, d, J8.85 Hz); 7.15 (5H, m); 3.81 (4H, m); 3.56 (3H, s); 3.21 (4H, m), LC-MS rt 2.62, m/z E+447

### Example 47

### (6-Iodo-quinazolin-4-yl)-(3-methyl-butyl)-(4-morpholin-4-yl-phenyl)-amine

Step 1: To a carousel tube was added N-(4-aminophenyl)morpholine (0.25 g, 1.40 mmol). DCM (DRY 15 ml), molecular sieve 3A (excess 0.2 g) and 3-methyl-butylaldehyde (0.14 g, 1.4 mmol). The mixture was stirred for 1 hr at room temperature and then at 45° C. for 2 hr. The mixture was cooled and acetic acid (1 ml) was added followed by sodium triacetoxy borohydride (0.60 g, 2.80 mmol) and the mixture was left to stir overnight at room temperature. The solvent was removed under vacuum and the crude product was purified by column chromatography on silica using 2.5% MeOH:DCM, to give (3-methyl-butyl)-(4-morpholin-4-yl-phenyl)-amine Yield 0.20 g (57%), δ (DMSO) 6.75 (2H, d, J8.85 Hz); 6.50 (2H, d, J8.85 Hz); 3.71 (4H, m); 3.17 (2H, m); 2.89 (4H, m); 1.66 (1H, m); 1.39 (2H, m); 0.91 (6H, d, J6.32 Hz), LCMS: RT 2.22, ES⁺ 249

Step 2: Treatment of (3-methyl-butyl)-(4-morpholin-4-yl-phenyl)-amine (210 mg) as per Example 47, step 3 gave the title compound (294 mg, 71%), δ(DMSO) 8.69 (1H, s); 7.89 (1H, dd, J8.85 Hz, 1.9 Hz); 7.50 (1H, d, J8.85 Hz); 7.16 (4H, AB, J8.85 Hz); 7.06 (1H, d, J1.90 Hz); 4.12 (2H, t, J7.58 Hz); 3.80 (4H, m); 3.23 (4H, m); 1.62 (2H, m); 1.37 (1H, m); 0.94 (6H, d, 6.32 Hz), LC-MS rt 3.11, m/z E+503

### Example 48

### Isopropyl-(4-morpholin-4-yl-phenyl)-(6-thiophen-2-yl-quinazolin-4-yl)-amine

By a similar method to Example 47 followed by a similar method to Example 45 step 4 was obtained the title compound (4.5 mg) δ(DMSO) 8.61 (1H, s); 7.90 (1H, d, J7.58 Hz); 7.67 (1H, d, J8.85 Hz); 7.51 (1H, d, J4.42 Hz); 7.09 (7H, m); 5.51 (1H, m); 3.77 (4H, m); 3.23 (4H, m); 1.18 (6H, d, J6.32 Hz), LC-MS rt 2.74, m/z E+431

### Example 49

### (3-Methyl-butyl)-(4-morpholin-4-yl-phenyl)-(6-thiophen-2-yl-quinazolin-4-yl)-amine

By a similar method to Example 44 step 4 was obtained the title compound (12.5 mg) δ(DMSO) 8.63 (1H,); 8.21 (1H, s); 7.94 (1H d, J8.85 Hz); 7.72 (2H, m); 7.52 (1H, d, J5.06 Hz); 7.12 (5H, m); 4.13 (2H, m); 3.78 (4H, m); 3.20 (4H, m); 3.0 (3H, m); 1.15 (6H, t, J7.58 Hz), LC-MS rt 2.98, m/z E+459

### Example 50

### [6-(2-Benzyloxy-phenyl)-quinazolin-4-yl]-(4-morpholin-4-yl-phenyl)-amine

By a similar method to Example 15 was obtained the title compound LC-MS m/z 489.4 rt 2.82

### Example 51

### [6-(4-Benzyloxy-phenyl)-quinazolin-4-yl]-(4-morpholin-4-yl-phenyl)-amine

By a similar method to Example 15 was obtained the title compound (DMSO, δ) 3.07-3.10 (t, 4H), 3.71-3.75 (t, 4H), 5.18 (s, 2H), 6.95-6.99 (d, 2H), 7.14-7.18 (d, 2H), 7.28-7.48 (m, 5H), 7.60-7.64 (d, 2H), 7.74-7.83 (m, 3H), 8.08-8.12 (d, 1H), 8.45 (s, 1H), 8.71 (s, 1H), LC-MS m/z 490 rt 2.89

### Activity Example

### Cells Used

HCV replicon cells Huh 9B (ReBlikon), containing the firefly luciferase—ubiquitin—neomycin phosphotransferase fusion protein and EMCV-IRES driven HCV polyprotein with cell culture adaptive mutations.

### Cell Culture Conditions:

Cells were cultured at 37° C. in a 5% CO₂ environment and split twice a week on seeding at 2×10E6 cells/flask on day 1 and 1×10E6 3 days later. Some 0.25 mg/ml G418 was added to the culture medium (125 ul per 25 ml) but not the assay medium.

The culture medium consisted of DMEM with 4500 g/l glucose and glutamax (Gibco 61965-026) supplemented with 1× non-essential amino acids, penicillin (100 IU/ml)/streptomycin (100 μg/ml), FCS (10%, 50 ml) and 1 mg/ml G418 (Invitrogen cat no 10131-027) & 10% foetal calf serum.

### Assay Procedure:

A flask of cells was trypsinised and a cell count carried out. Cells were diluted to 100,000 cells/ml and 100 μl of this used to seed one opaque white 96-well plate (for the replicon assay) and one flat-bottomed clear plate (for the tox assay) for every seven compounds to be tested for IC50. Wells G12 and H12 were left empty in the clear plate as the blank. Plates were then incubated at 37° C. in a 5% CO₂ environment for 24 h.

On the following day compound dilutions are made up in medium at twice their desired final concentration in a clear round bottomed plate. All dilutions have a final DMSO concentration of 1%.

Once the dilution plate had been made up, controls and compounds were transferred to the assay plate (containing the cells) at 100 μl/well in duplicate plates.

Exception: in the white (replicon) plate, no compound was added to wells A1 and A2 and 100 μl of 1% DMSO was added to these instead. In the clear (Tox) plate, wells E12 & F12 only contained the DMSO control. Plates were then incubated at 37° C. with 5% CO₂ for 72 h.

At the end of the incubation time, the cells in the white plate were harvested by washing with 200 μl/well of warm (37° C.) PBS and lysed with 20 μl cell culture lysis buffer (Promega). After 5 min incubation @ RT, luciferin solution was added to the luciferase assay buffer (LARB at 200 μl per 10 ml LARB. The M injector of the microplate luminometer (Lmax, Molecular Devices) was primed with 4×300 l injections. Plate were inserted into the luminometer and 100 μl luciferase assay reagent was added by the injector on the luminometer. The signal was measured using a 1 second delay followed by a 4 second measurement programme. The IC50, the concentration of the drug required for reducing the replicon level by 50% in relation to the untreated cell control value, can be calculated from the plot of the percentage reduction of the luciferase activity vs. drug concentration.

The clear plate was stained with 100 μl 0.5% methylene blue in 50% ethanol at RT for 1 h, followed by salvation of the absorbed methylene blue in 100 μl per well of 1% lauroylsarcosine. Absorbance of the plate was measured on a microplate spectrophotometer (Molecular Devices) and the absorbance for each concentration of compound expressed as a proportion of the relative DMSO control. The TD50, the concentration of drug required to reduce the total cell area by 50% relative to the DMSO controls can be calculated by plotting the absorbance at 620 nm vs drug concentration.

TABLE 1

| | Replicon IC50 | Replicon TD50 |
|---|---|---|
| | (<1 uM = ***; <5 | (>25 uM = ***; >10 |
| | uM = **; <25 | uM = **; >1 |
| Example | uM = *) | uM = *) |
| no | uM | uM |
| 1 | ** | *** |
| 2 | *** | ** |
| 3 | * | *** |
| 4 | * | *** |
| 5 | *** | * |
| 6 | ** | ** |
| 7 | *** | ** |
| 8 | *** | *** |
| 9 | *** | ** |
| 10 | ** | *** |
| 11 | ** | *** |
| 12 | *** | *** |
| 13 | ** | ** |
| 14 | * | *** |
| 15 | *** | *** |
| 16 | ** | *** |
| 17 | ** | *** |
| 18 | *** | *** |
| 19 | *** | *** |
| 20 | ** | *** |
| 21 | * | *** |
| 22 | *** | *** |
| 23 | *** | ** |
| 24 | ** | *** |
| 25 | * | *** |
| 26 | ** | *** |
| 27 | ** | *** |
| 28 | ** | *** |
| 29 | ** | *** |
| 30 | ** | *** |
| 31 | ** | *** |
| 32 | ** | *** |
| 33 | ** | *** |
| 34 | *** | *** |
| 35 | *** | *** |
| 36 | *** | *** |
| 37 | ** | ** |
| 38 | ** | *** |
| 39 | *** | *** |
| 40 | *** | ** |
| 41 | ** | ** |
| 42 | *** | *** |
| 43 | *** | ** |
| 44 | *** | *** |
| 45 | ** | *** |
| 46 | *** | * |
| 47 | ** | *** |
| 48 | ** | *** |
| 49 | ** | ** |
| 50 | ** | *** |
| 51 | ** | *** |

1. A compound which is a quinazoline derivative of formula (Ia), or a pharmaceutically acceptable salt thereof, 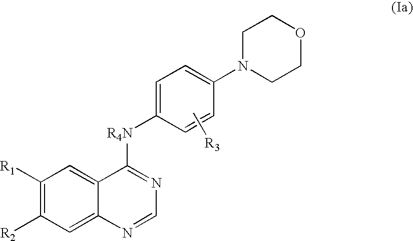
wherein
R₁ represents hydrogen, halogen, C₁-C₄ alkyl, C₁-C₄ haloalkyl, C₁-C₄ alkoxy, C₁-C₄ haloalkoxy, —CO₂R′, —CONR′R″, -A, -A-L-A′, -Z-L-A or -A-L-Z-L-A, wherein R′ and R″ are the same or different and each represent hydrogen or C₁-C₄ alkyl;
R₂ represents hydrogen, halogen, C₁-C₄ alkyl, C₁-C₄ haloalkyl, C₁-C₄ alkoxy or C₁-C₄ haloalkoxy;
R₃ represents hydrogen, C₁-C₄ alkyl, C₁-C₄ haloalkyl, C₁-C₄ alkoxy or C₁-C₄ haloalkoxy; and
R₄ represents hydrogen, C₁-C₆ alkyl or C₁-C₆ haloalkyl,
wherein:
A represents a C₆ to C₁₀ aryl, 5- to 10-membered heteroaryl or 5- to 10 membered heterocyclyl group;
each L is the same or different and is a direct bond or a C₁-C₄ alkylene group;
A′ is a 5- to 10-membered heteroaryl or 5- to 10-membered heterocyclyl group; and
Z is —S—, —O—, —NR′—, —CO₂—, —C(O)NR′—, —OC(O)—, —NR′C(O)—, —OCO₂—, —NR′CO₂—, —OC(O)NR′—, or —NR′C(O)NR″—, wherein R′ and R″ are the same or different and represent hydrogen or C₁-C₄ alkyl,
the aryl, heteroaryl and heterocyclyl moieties in R₁ being unsubstituted or substituted by 1, 2 or 3 substituents selected from halogen, C₁-C₄ alkyl, C₁-C₄ haloalkyl, C₁-C₄ haloalkoxy, hydroxy, thiol, —NH₂, C₁-C₄ hydroxyalkyl, C₁-C₄ thioalkyl, C₁-C₄ aminoalkyl, cyano, nitro, —COR′, —CO₂R′, —S(O)R′, —S(O)₂R′, —CONR′R″ and -L′-X-L″-Y substituents, wherein each R′ and R″ is the same or different and is selected from hydrogen and C₁-C₄ alkyl, L′ is a direct bond or a C₁-C₄ alkylene group, X is —S—, —O— or —NR′— wherein R′ is as defined above, L″ is a direct bond or a C₁-C₄ alkylene group and Y is hydrogen, —COR′, —CO₂R′, —S(O)₂R′ or —S(O)R′, wherein R′ is hydrogen or C₁-C₄ alkyl.

2. A compound according to claim 1, wherein L′ is a direct bond or a C₁-C₂ alkylene group.

3. A compound according to claim 1, wherein X is —O— or —NR′— wherein R′ is as defined in claim 1.

4. A compound according to claim 1, wherein L″ is a direct bond or a C₁-C₂ alkylene group.

5. A compound according to claim 1, wherein Y is hydrogen, —COR′, —CO₂R′, —S(O)R′ or —S(O)₂R′, wherein R′ is a C₁-C₄ alkyl group.

6. A compound according to claim 1, wherein the aryl, heteroaryl and heterocyclyl moieties in the R₁ substituent are unsubstituted or substituted by 1, 2 or 3 substituents selected from halogen, C₁-C₄ alkyl, C₁-C₄ haloalkyl, C₁-C₄ haloalkoxy, C₁-C₄ hydroxyalkyl, cyano, —COR′—CO₂R′, —S(O)R′, —S(O)₂R′, and
-L′-X-L″-Y substituents, wherein R′, L′, X, L″ and Y are as defined in any one of the preceding claims.

7. A compound according to claim 1, wherein the aryl, heteroaryl and heterocyclyl moieties in the R₁ substituent are unsubstituted or substituted with 1 or 2 substituents selected from halogen, C₁-C₂ alkyl C₁-C₂ haloalkyl, C₁-C₂ hydroxyalkyl, cyano, —COR′, —CO₂R′, —S(O)R′, —S(O)₂R′, —(C₁-C₂ alkyl)-NR′R″, C₁-C₂ alkoxy, —NR′—COR′, NR′—CO₂R′, —(C₁-C₂ alkyl)-NR′—CO₂R′, —NR′—S(O)₂—R′ and
—(C₁-C₂ alkyl)-NR′—(C₁-C₂ alkyl)-S(O)₂—R″ substituents, wherein each R′, R″ and R′ are the same or different and represent hydrogen or C₁-C₂ alkyl.

8. A compound according to claim 1, wherein A is a phenyl, 5- to 6-membered heteroaryl or 5- to 6-membered heterocyclyl group.

9. A compound according to claim 1, wherein A is a phenyl, furanyl, thienyl, pyrimidinyl, thiazolyl or pyridazolyl group.

10. A compound according to claim 1, wherein L is a direct bond or a C₁-C₂ alkylene group.

11. A compound according to claim 1, wherein A′ is a 5- to 6-membered heterocyclyl or heteroaryl group which is unsubstituted or substituted with 1, 2 or 3 substituents selected from halogen, C₁-C₄ alkyl, C₁-C₄ haloalkyl and C₁-C₄ haloalkoxy substituents.

12. A compound according to claim 1, wherein A′ is a morpholinyl, thiomorpholinyl, piperazinyl, 1,3-dioxolanyl, S,S-dioxothiomorpholinyl or pyrazolyl group which is unsubstituted or substituted by one or two substituents selected from C₁-C₂ alkyl, halogen and C₁-C₂ haloalkyl substituents.

13. A compound according to claim 1, wherein Z is —O—, —CONR′—, —NR′C(O)— or —NR′CO₂—, wherein R′ is as defined in any preceding claim.

14. A compound according to claim 1, wherein Z is —O—, —CONH— or —CON(C₁-C₂ alkyl)- or —NHC(O)—, —NHCO₂—.

15. A compound according to claim 1, wherein R₁ is halogen, C₁-C₄ alkyl, C₁-C₄ haloalkyl, C₁-C₄ alkoxy, C₁-C₄ haloalkoxy, —CO₂R′, —CONR′R″, -A, -A-L-A′, -Z-L-A, or -A-L-Z-L-A wherein R′, R″, A, L, A′ and Z are as defined in any one of the preceding claims.

16. A compound according to claim 1, wherein R₁ is halogen, C₁-C₂ alkoxy, C₁-C₂ haloalkoxy, —CONR′R″, -A, —Ar-L-A′, -Z-L-A or —Ar-Z-L-Ar, wherein R′ and R″ are the same or different and each represent hydrogen or a C₁-C₂ alkyl group, A and A′ are as defined in any one of the preceding claims, Ar is an unsubstituted furanyl or unsubstituted phenyl group, L is a direct bond or a methylene group and Z is —O—, —C(O)NR′—, —NR′C(O)— or —NR′CO₂—, wherein R′ is hydrogen or a C₁-C₄ alkyl group.

17. A compound according to claim 1, wherein R₂ is hydrogen, C₁-C₄ alkyl or C₁-C₄ alkoxy.

18. A compound according to claim 1, wherein R₃ is hydrogen, C₁-C₂ alkyl, C₁-C₂ haloalkyl or C₁-C₂ alkoxy.

19. A compound according to claim 1, wherein R₄ is hydrogen or C₁-C₆ alkyl.

20. A compound according to claim 1, wherein the quinazoline derivative of formula (Ia) is a quinazoline derivative of formula (I), 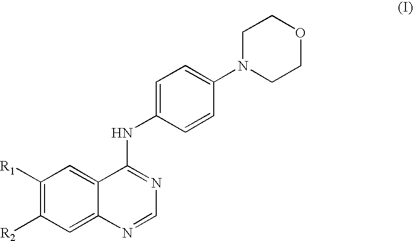
wherein:
R₁ is halogen, C₁-C₂ alkoxy, C₁-C₂ haloalkoxy, -A or —Ar-L-A′;
R₂ is hydrogen or C₁-C₂ alkoxy;
A is a phenyl or 5- to 6-membered heteroaryl group, for example furanyl, thienyl, pyrimidinyl and thiazolyl, which group is unsubstituted or substituted with 1 or 2 substituents selected from halogen, C₁-C₂ alkyl, C₁-C₂ alkoxy, C₁-C₂ haloalkyl, C₁-C₂ hydroxyalkyl, —COR′, —CO₂R′, —S(O)R′, —S(O)₂R′, —(C₁-C₂ alkyl)-NR′R″ and —(C₁-C₂ alkyl)-NR′—(C₁-C₂ alkyl)-S(O)₂—R″ substituents, wherein each R′ and R″ are the same or different and represent hydrogen or —C₁-C₂ alkyl;
Ar is an unsubstituted furanyl group;
L is a direct bond or a methylene group; and
A′ is a 5- to 6-membered heterocyclyl group, for example morpholinyl, thiomorpholinyl, piperazinyl, 1,3-dioxoanyl and S,S-dioxothiomorpholinyl, which is unsubstituted or substituted by one or two substituents selected from C₁-C₂ alkyl, halogen and C₁-C₂ haloalkyl groups.

21. A compound according to claim 20, wherein:
R₁ is halogen, C₁-C₂ alkoxy, C₁-C₂ haloalkoxy, -A or —Ar-L-A′;
R₂ is hydrogen or C₁-C₂ alkoxy;
A is a phenyl or 5- to 6-membered heteroaryl group, for example furanyl, thienyl and thiazolyl, which group is unsubstituted or substituted with 1 or 2 substituents selected from halogen, C₁-C₂ alkyl, C₁-C₂ haloalkyl, C₁-C₂ hydroxyalkyl, —COR′, —(C₁-C₂ alkyl)-NR′R″ and —(C₁-C₂ alkyl)-NR′—(C₁-C₂ alkyl)-S(O)₂—R″ substituents, wherein each R′ and R″ are the same or different and represent hydrogen or C₁-C₂ alkyl;
Ar is an unsubstituted furanyl group;
L is a direct bond or a methylene group; and
A′ is a 5- to 6-membered heterocyclyl group, for example morpholinyl, thiomorpholinyl, piperazinyl, 1,3-dioxoanyl and S,S-dioxothiomorpholinyl, which is unsubstituted or substituted by one or two substituents selected from C₁-C₂ alkyl, halogen and C₁-C₂ haloalkyl groups.

22. (canceled)

23. A pharmaceutical composition which comprises a quinazoline derivative of the formula (Ia), as defined in claim 1, or a pharmaceutically acceptable salt thereof, and a pharmaceutically acceptable carrier or diluent.

24. A method of alleviating or reducing the incidence of a flaviviridae infection in a patient which method comprises administering to said patient an effective amount of a quinazoline derivative of the formula (Ia), as defined in claim 1, or a pharmaceutically acceptable salt thereof.

25. A method according to claim 24, wherein the flaviviridae infection is a pestivirus infection.

26. A method according to claim 25, wherein the pestivirus infection is an infection by a bovine viral diarrhea virus, classical swine fever virus or border disease virus.

27. A method according to claim 24, wherein the flaviviridae infection is a flavivirus infection.

28. A method according to claim 27, wherein the flavivirus infection is an infection by a yellow fever virus, dengue fever virus, Japanese encephalitis virus or tick borne encephalitis virus.

29. A method according to claim 24, wherein the flaviviridae infection is a hepacivirus infection.

30. A method according to claim 29, wherein the hepacivirus infection is an infection by a hepatitis C virus.

31. A method according to claim 30, wherein (a) interferon or a derivative thereof and/or (b) ribavirin or a derivative thereof is also administered to the patient.

32. A method according to claim 31 wherein the interferon derivative is PEG-interferon and/or the ribavirin derivative is viramidine.

33. (canceled)

34. (canceled)
